(19) United States
(12) Patent Application Publication
(10) Pub. No.: US 2026/0260698 A1
(43) Pub. Date: Sep. 3, 2026

(54) VARIANT CALLING WITH METHYLATION-LEVEL ESTIMATION

(71) Applicant: Illumina, Inc., San Diego, CA (US)

(72) Inventors: JAMES BAYE, Cambridge (GB); DANIEL ANDREWS, Cambridge (GB); KONRAD HAARHOFF SCHEFFLER, Cambridge (GB)

(21) Appl. No.: 19/491,221

(22) PCT Filed: Jun. 26, 2024
(86) PCT No.: PCT/US2024/035562

§ 371 Date: Dec. 8, 2025

Related U.S. Application Data
(60) Provisional application No. 63/510,603, filed on Jun. 27, 2023.

(51) Int. Cl. G16B 20/20; G16B 40/00

(52) U.S. Cl. CPC G16B 20/20; G16B 40/00

(57) ABSTRACT

This disclosure describes methods, non-transitory-computer readable media, and systems that can simultaneously determine estimated methylation-level values for cytosine bases and genotype calls for a target genomic sample. The disclosed system can utilize a Bayesian method on a target genomic sample's nucleotide-read data to generate estimated methylation-level values that indicate genomic coordinates at which the target genomic sample comprises a reference cytosine base or a nucleobase that could be called as a cytosine. The disclosed system can estimate methylation-level values based on prior genotype probabilities and observed nucleobases at a genomic coordinate from a read pileup of a target genomic sample. Based on the estimated methylation-level values and base-call-quality metrics, the disclosed system may generate posterior genotype probabilities for the genomic sample at the genomic coordinate. Based on the posterior genotype probabilities, the disclosed system can generate a genotype call for the target genomic sample.

### CROSS-REFERENCE TO RELATED APPLICATIONS

This application claims priority to and the benefit of U.S. Provisional Patent Application No. 63/510,603, entitled “VARIANT CALLING WITH METHYLATION-LEVEL ESTIMATION” filed Jun. 27, 2023, which is incorporated herein by reference in its entirety.

### BACKGROUND

In recent years, biotechnology firms and research institutions have improved hardware and software for sequencing nucleotides and determining nucleobase calls for genomic samples. For instance, some existing sequencing machines and sequencing-data-analysis software (together “existing sequencing systems”) predict individual nucleobases within sequences by using conventional Sanger sequencing or sequencing-by-synthesis (SBS) methods. When using SBS, existing sequencing systems can monitor many thousands to millions of oligonucleotides being synthesized in parallel from templates to predict nucleobase calls for growing nucleotide reads. In many existing sequencing systems, a camera captures images of irradiated fluorescent tags incorporated into oligonucleotides. After capturing such images, some existing sequencing systems determine nucleobase calls for nucleotide reads corresponding to the oligonucleotides and send base-call data to a computing device with sequencing-data-analysis software, which aligns nucleotide reads with a reference genome. Based on differences between the aligned nucleotide reads and the reference genome, existing systems can further utilize a variant caller to identify variants of a genomic sample, such as single nucleotide variants (SNVs), insertions or deletions (indels), or other variants within the genomic sample.

In addition to improved genomic sequencing, biotechnology firms and research institutions have also improved methods of detecting methylation of cytosine bases at particular genomic regions (e.g., regions encoding or promoting genes) and detecting methylation of larger nucleotide fragments or whole genomes of a sample. For instance, some existing sequencing systems can use sequencing devices and corresponding sequencing-data-analysis software to identify when a methyl or hydroxymethyl group has been added to a cytosine base of a sample's deoxyribonucleic acid (DNA)—where the methylated cytosine base is often part of a cytosine-guanine-dinucleotide pair in a 5′-C-phosphate-G-3′ (CpG) configuration in mammals. For example, existing sequencing systems can detect methylated cytosines by (i) enzymatically converting methylated or unmethylated cytosine bases at CpG or other cytosine sites from a sample nucleotide fragment into uracil bases (e.g., dihydrouracil); (ii) determining base calls of nucleotide reads for the sample using a sequencing device, where the sequencing device detects the uracil bases as thymine bases during polymerase chain reaction (PCR) amplification; and (iii) comparing the base calls from the nucleotide reads to a reference genome or non-enzymatically converted nucleotide reads from the sample. Based on the comparison of nucleotide reads from the sample to a reference genome or the non-enzymatically converted nucleotide reads, existing sequencing systems can identify thymine bases from the nucleotide reads that do not match cytosine bases at CpG or other cytosine sites within the reference genome or the non-enzymatically converted nucleotide reads and thereby detect methylated cytosine bases in a sample nucleotide fragment.

Despite these recent advances, existing sequencing and methylation detection systems face several technical shortcomings. Different types of methylation assays detect methylated cytosines by converting methylated or unmethylated cytosine bases into uracil bases and subsequently, in some cases, into thymine bases. As oligonucleotides extracted from the genomic sample are duplicated as part of the methylation sequencing assay, complementary strands reflect regions of cytosine-to-thymine substitutions by having adenines in place of guanines. While these conversions aid in the detection of methylation, the conversions may also negatively affect performance and accuracy of existing sequencing systems.

For example, existing sequencing and methylation detection systems cannot consistently generate accurate genotype calls when simultaneously calling genotype and methylation level when performing C>T conversion-based sequencing. Due in part to C>T conversions in methylation assays, existing sequencing systems often produce biased genotype calls and biased methylation-level estimates. Converted methylated or unmethylated cytosine bases often introduce noise into sequence data that, in turn, hinders accurate variant calling. Because of such conversions and noise in methylation assays, existing methylation detection systems often overestimate methylation levels for C/A, C/T, G/A, and G/T genotypes. Furthermore, existing sequencing systems frequently determine inaccurate base calls for genomic regions comprising converted methylated or unmethylated cytosine bases. For example, because of noise in sequence data, existing sequencing systems often falsely call methylation events as C/T or G/A genotypes.

As noted above, in addition to biased genotype calls, existing sequencing systems generate biased methylation-level estimates when concurrently determining genotype. Existing sequencing systems often rely on overly simplistic methods or algorithms to estimate methylation levels. For example, some existing systems attempt to improve the accuracy of simultaneous methylation-level estimation and genotype calling by correcting data, ignoring data, or creating statistical models that account for difficult-to-call genomic regions. As a result, existing systems are often subject to coding errors and yield limited benefits in accuracy.

In addition to accuracy challenges, some existing sequencing and methylation detection systems inefficiently consume an inordinate amount of processing materials, time, and computing resources. Existing sequencing and methylation detection systems often require multiple assays and samples to accurately determine methylation levels and variant calls. For example, existing systems often require a separate methylation assay in addition to the utilization of a separate variant caller to accurately determine both methylation-level values and genotype calls. Accordingly, some existing systems require multiple samples from a single organism on which to perform both sequencing and methylation assays in separate computational analyses. The duplication of genomic samples often necessitates a duplication of computer processing, computer storage, software programs, and other resources to sequence and determine methylation levels for the same genomic sequence. Thus, existing systems often consume excessive genomic samples, significant time, and computer processing resources to both sequence and determine methylation levels for a single genomic sequence.

These, along with additional problems and issues exist in existing sequencing and methylation detection systems.

### SUMMARY

This disclosure describes one or more embodiments of systems, methods, and non-transitory computer readable storage media that solve one or more of the problems described above or provide other advantages over the art. For example, the disclosed systems accurately and simultaneously determine estimated methylation-level values for cytosine bases and genotype calls for a target genomic sample by utilizing a Bayesian method on the target genomic sample's nucleotide-read data. Such estimated methylation-level values can include genomic coordinates at which the target genomic sample comprises a reference cytosine bases or a nucleobase that could be called as cytosine (e.g., cytosine base on a minus strand). In particular, the disclosed system can estimate methylation-level values based on prior genotype probabilities and observed nucleobases at a genomic coordinate from a read pileup of a target genomic sample. Based on the estimated methylation-level values and base-call-quality metrics, the disclosed system may generate posterior genotype probabilities for the genomic sample at the genomic coordinate. From such posterior genotype probabilities, the disclosed system generates a genotype call for the target genomic sample. In some implementations, the disclosed system further refines the estimated methylation-level values to generate a more accurate, refined methylation-level values for the target genomic sample based on the posterior genotype probabilities and observed nucleobases from the read pileup.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The detailed description refers to the drawings briefly described below.

FIG. 1 illustrates an environment in which a methylation-genotype-calling system can operate in accordance with one or more embodiments of the present disclosure.

FIGS. 2A-2B illustrate an overview of the methylation-genotype-calling system simultaneously determining genotype calls and methylation-level values for observed bases from a read pileup in accordance with one or more embodiments of the present disclosure.

FIG. 3 illustrates various types of methylation sequencing protocols that the methylation-genotype-calling system may utilize in accordance with one or more embodiments of the present disclosure.

FIG. 4 illustrates the methylation-genotype-calling system accurately estimating genotype and methylation-level values based on observed nucleobases in accordance with one or more embodiments of the present disclosure.

FIG. 5 illustrates a model employed by existing sequencing and methylation detection systems that inaccurately estimate methylation-level values.

FIG. 6 illustrates graphs demonstrating estimated methylation-level values by existing sequencing systems compared with true methylation-level values for a set of genotypes.

FIGS. 7A-7B illustrate a series of acts by which the methylation-genotype-calling system determines an estimated methylation-level value for a cytosine base in accordance with one or more embodiments of the present disclosure.

FIG. 8 illustrates an overview of the methylation-genotype-calling system generating posterior genotype probabilities in accordance with one or more embodiments of the present disclosure.

FIG. 9 illustrates the methylation-genotype-calling system determining the probability of an observed base and posterior genotype probabilities based in part on estimated methylation-level values in accordance with one or more embodiments of the present disclosure.

FIG. 10 illustrates the methylation-genotype-calling system generating a refined methylation-level value in accordance with one or more embodiments of the present disclosure.

FIGS. 11A-11B illustrate graphs demonstrating improvements made by the methylation-genotype-calling system in accurately predicting methylation-level values in both germline and somatic alleles in accordance with one or more embodiments of the present disclosure.

FIGS. 12A-12B illustrate a series of plots demonstrating that the methylation-genotype-calling system more accurately calls single nucleotide polymorphisms (SNPs) in accordance with one or more embodiments of the present disclosure.

FIGS. 13A-13C illustrate a series of charts demonstrating that the methylation-genotype-calling system accurately calls genotypes at genomic coordinates in accordance with one or more embodiments of the present disclosure.

FIG. 14 illustrates a flowchart of a series of acts of determining an estimated methylation-level value and generating a genotype call in accordance with one or more embodiments of the present disclosure.

FIG. 15 illustrates a block diagram of an example computing device in accordance with one or more embodiments of the present disclosure.

### DETAILED DESCRIPTION

This disclosure describes one or more embodiments of a methylation-genotype-calling system that can accurately determine genotype and methylation levels for a genomic sample from the genomic sample's nucleotide-read data. For instance, the methylation-genotype-calling system can access, for a target genomic sample, nucleotide reads comprising nucleobases that have been converted by a methylation sequencing assay. The methylation-genotype-calling system can further estimate a methylation level of a candidate cytosine base at a genomic coordinate based on prior genotype probabilities and observed nucleobases at the genomic coordinate within the nucleotide reads. The methylation-genotype-calling system further utilizes a variant call model to generate posterior genotype probabilities for the target genomic sample at the genomic coordinate based on the estimated methylation level and base-call-quality metrics for the observed nucleobases. Based on the posterior genotype probabilities, the methylation-genotype-calling system can predict a genotype call at the genomic coordinate for the target genomic sample. In some implementations, the methylation-genotype-calling system further refines and increases an accuracy of the estimated methylation-level value for the target genomic sample based on the posterior genotype probabilities.

As just noted, the methylation-genotype-calling system can identify, for a target genomic sample, nucleotide reads comprising one or more nucleobases converted by a methylation sequencing assay. As part of identifying converted nucleobases, the methylation-genotype-calling system identifies, from within the identified nucleotide reads, genomic coordinates comprising candidate cytosine bases within the target genomic sample that may be methylated. In some implementations, the methylation-genotype-calling system identifies genomic coordinates comprising alternative haplotypes of cytosine bases. The methylation-genotype-calling system can identify candidate cytosine bases within a target genomic sample, even if they do not match a reference genome. More particularly, the methylation-genotype-calling system identifies genomic coordinates of a reference genome or a target genomic sample containing a cytosine base, including, but not limited to, (i) genomic coordinates where the reference base is cytosine base and the called genotype contains a cytosine base or (ii) genomic coordinates in the target genomic sample where the reference base is not a cytosine base (e.g., ref=A) but the called genotype contains a cytosine base.

Having identified genomic coordinates comprising alternative haplotypes, such as (i) and (ii) genomic coordinates comprising candidate cytosine bases, the methylation-genotype-calling system can determine an estimated methylation-level value for a candidate cytosine base at a genomic coordinate based on prior genotype probabilities for the target genomic sample and based on observed nucleobases within the nucleotide reads. For example, the methylation-genotype-calling system can generate an estimated methylation-level value (a) for each genomic coordinate at which the reference base is a cytosine base and the prior genotype probability indicates a cytosine base and (b) for each genomic coordinate at which the target genomic sample comprises cytosine bases as alternative haplotypes. In particular, the methylation-genotype-calling system determines the probability of a given, observed read pileup based on the prior genotype probabilities from observed nucleobases and probabilities for each nucleobase at the genomic coordinate within the read pileup on both plus and minus strands.

For the prior genotype probabilities, the methylation-genotype-calling system assumes different prior genotype probabilities for each nucleobase as a basis for determining estimated methylation-level values. For example, the methylation-genotype-calling system can determine that haplotypes not matching cytosines are not methyl-converted. As described further below, in some cases, the methylation-genotype-calling system assumes that (i) the prior probability of a thymine base on a plus strand is approximately equal to a beta value, where the beta value represents a position-specific fraction of cytosine bases methyl-converted to thymine bases, (ii) the prior probability of a cytosine base on the plus strand is approximately equal to the formula 1 minus the beta value, and (iii) the prior probability of an adenine or guanine base on the plus strand is determined by the base-call-error probability (e.g., a base-call error over 3). Conversely, the prior probability of an adenine, guanine, or thymine base on the minus strand is likewise determined by the base-call-error probability. Further, the prior probability of cytosine on the plus strand is approximately equal to the formula 1 minus the base-call-error probability. The methylation-genotype-calling system may then generate the estimated methylation-level value for a cytosine base by performing a Bayesian inversion on the prior probabilities exhibited by observed nucleobases within a given read pileup.

Based on the estimated methylation-level value and base-call-quality metrics (e.g., Q-score) and/or other sequencing metrics for the observed nucleobases, in some embodiments, the methylation-genotype-calling system utilizes a variant call model to generate posterior genotype probabilities for the target genomic sample at the genomic coordinate. More specifically, the methylation-genotype-calling system determines an input representing the estimated methylation-level value and the base-call-quality metrics and feeds the input into a variant call model modified to receive such methylation-level-derived inputs. The methylation-genotype-calling system utilizes the variant call model to generate posterior genotype probabilities and further determines a highest posterior genotype probability as the genotype call.

In some implementations, the methylation-genotype-calling system further utilizes the posterior genotype probabilities to generate a refined methylation-level value for a nucleobase at a genomic coordinate. The refined methylation-level value can represent a cytosine methylation percentage at the genomic coordinate. More specifically, the methylation-genotype-calling system can determine refined methylation-level values (a) for genomic coordinates at which the reference base is a cytosine base and the prior genotype probability indicates a cytosine base and (b) for genomic coordinates at which the target genomic sample comprises cytosine bases as alternative haplotypes. The methylation-genotype-calling system may determine the refined methylation-level value based on posterior genotype probabilities, a number of reads in the read pileup, and a number of methylated nucleobases in the read pileup. Because the methylation-genotype-calling system determines the refined methylation-level value based on posterior genotype probabilities, the refined methylation-level value may be more accurate than the estimated methylation-level value.

As indicated above, the methylation-genotype-calling system provides several technical advantages relative to existing sequencing systems by, for example, improving methylation and genotype calling accuracy and computational efficiency relative to existing sequencing systems. As mentioned, the methylation-genotype-calling system improves the accuracy of methylation-level value and genotype calling relative to existing sequencing systems. The methylation-genotype-calling system more accurately identifies potential methylation positions in a target genomic sample. As mentioned, existing systems often fail to estimate methylation at genomic coordinates of a target genomic sample that do not align with cytosine bases within a reference genome. In contrast, the methylation-genotype-calling system estimates methylation-level values for cytosine bases identified within the target genomic sample and not just for positions at which the target genomic sample comprises a cytosine base matching the reference genome. As indicated below, the methylation-genotype-calling system exhibits a precision and recall for germline and somatic SNV calling that approaches the accuracy of non-methylated whole genome sequencing.

The methylation-genotype-calling system also more accurately estimates methylation levels for cytosine bases within a target genomic sample. Because the methylation-genotype-calling system accounts for bases that are methyl-converted (e.g., cytosine and thymine) on both the plus and minus strands of the target genomic sample, the methylation-genotype-calling system generates more accurate estimated methylation-level values for such bases. The methylation-genotype-calling system may utilize the estimated methylation-level values to generate more accurate genotype calls than existing methylation-read-based variant callers. Furthermore, in some implementations, the methylation-genotype-calling system can generate refined methylation-level values that improve accuracy over the initially determined methylation-level values by leveraging posterior genotype probabilities used to determine the genotype calls.

Beyond improved genotype and methylation call accuracy, in some embodiments, the methylation-genotype-calling system improves efficiency in processing and physical resources relative to existing systems. As noted above, because state-of-the-art genotype-calling and methylation detection accuracy can be unfit for clinical benchmarks, some existing systems execute (i) a separate methylation sequencing assay to chemically or enzymatically convert nucleotide reads from a genomic sample and determine methylation levels and (ii) a separate DNA sequencing run with non-chemically or non-enzymatically converted nucleotide reads from the genomic sample to determine variant calls. Such separate methylation sequencing assays and DNA sequencing can consume and duplicate computer processing, memory storage, physical space and reagents for a nucleotide-sample slide (e.g., flow cell), and software programs (e.g., separate methylation analysis and variant calling software). In contrast to such a bifurcated approach, in some embodiments, the methylation-genotype-calling system can simultaneously determine methylation-level values indicating levels of methylation of a target genomic sample's cytosine bases and generate variant calls for the genomic sample with improved accuracy. Thus, the methylation-genotype-calling system can efficiently generate epigenetic and genetic sequencing data from a single genomic sample. By generating both methylation-level values and variant calls from the same genomic sample, the methylation-genotype-calling system further reduces the amount of computer processing, computer storage, software programs, space used on a nucleotide-sample slide in a sequencing device, and other resources to generate accurate sequencing and methylation data.

As illustrated by the foregoing discussion, the present disclosure utilizes a variety of terms to describe features and advantages of the methylation-genotype-calling system. As used herein, for example, the term “methylation sequencing assay” refers to an assay that detects, measures, or quantifies methylation of cytosine from an oligonucleotide or other nucleotide sequence. In some cases, a methylation sequencing assay detects or quantifies methylation of cytosine at particular target genomic regions or in particular cell types. Some methylation sequencing assays quantify methylation in terms of methylation-level values.

Relatedly, the term “methylation-level value” refers to a numeric value indicating an amount, percentage, ratio, or quantity of cytosine to which a methyl group or hydroxymethyl group has been added or bonded. For instance, a methylation-level value includes a score (e.g., ranging from 0 to 1) that indicates a percentage or ratio of cytosine bases (e.g., at CpG or other cytosine sites) for particular genomic coordinates or genomic regions to which a methyl group has been added. In some cases, a methylation-level value is expressed as a beta value (β) or an M value. To illustrate, a beta value may estimate a methylation level using a ratio of signal intensities between methylated alleles corresponding to a genomic coordinate and unmethylated alleles corresponding to the genomic coordinate, where 0 represents completely unmethylated and 1 represents completely methylated. In another example, a beta value may comprise a genomic-coordinate-specific fraction of cytosine bases that are methyl-converted to thymine bases. By contrast, an M value may represent a log 2 ratio of signal intensities of a methylated probe and an unmethylated probe corresponding to a cytosine base. As described below, the disclosed methylation-genotype-calling system can determine strand-specific methylation-level values, such as a first methylation-level value for a nucleobase at a genomic coordinate on a plus strand and a second methylation-level value for a nucleobase at the genomic coordinate on a minus strand.

As used herein, the term “refined methylation-level value” refers to a modified or updated methylation-level value based on new or previously unavailable data (e.g., posterior genotype probabilities). For instance, a refined methylation-level value includes a score (e.g., ranging from 0 to 1) that indicates a percentage or ratio of cytosine bases (e.g., at CpG or other cytosine sites) for particular genomic coordinates or genomic regions to which a methyl group has been added. In some cases, a refined methylation-level value is expressed as a beta value (\hat{β}) or an M value. Furthermore, in some embodiments, the methylation-genotype-calling system generates a refined methylation-level value that is more accurate than an initial estimated methylation-level value. For example, the methylation-genotype-calling system can utilize posterior genotype probabilities and observed nucleobases to generate the refined methylation-level value.

As used herein, the term “variant call model” (or simply “variant caller”) refers to a probabilistic model that generates rapid sequencing data from nucleotide reads of a sample nucleotide sequence, including variant calls and associated metrics. For example, in some cases, a variant call model refers to a Bayesian probability model that generates variant calls based on nucleotide reads of a sample nucleotide sequence. Such a model can process or analyze sequencing metrics corresponding to read pileups (e.g., multiple nucleotide reads corresponding to a single genomic coordinate), including mapping quality, base quality, and various hypotheses including foreign reads, missing reads, joint detection, and more. A variant call model may likewise include multiple components, including, but not limited to, different software applications or components for mapping and aligning, sorting, duplicate marking, computing read pileup depths, and variant calling. In some cases, the variant call model refers to the ILLUMINA DRAGEN model for variant calling functions and mapping and alignment functions.

As further used herein, the term “nucleotide read” (or simply “read”) refers to an inferred sequence of one or more nucleobases (or nucleobase pairs) from all or part of a sample nucleotide sequence (e.g., a sample genomic sequence, complementary DNA). In particular, a nucleotide read includes a determined or predicted sequence of nucleobase calls for a nucleotide sequence (or group of monoclonal nucleotide sequences) from a sample library fragment corresponding to a genomic sample. For example, in some cases, a sequencing device determines a nucleotide read by generating nucleobase calls for nucleobases passed through a nanopore of a nucleotide-sample slide, determined via fluorescent tagging, or determined from a cluster in a flow cell.

As used herein, the term “nucleobase” refers to a nitrogenous base. In particular, nucleobases comprise components of nucleotides. For example, a nucleobase may be an adenine (A), cytosine (C), guanine (G), or thymine (T).

As used herein, the term “observed nucleobase” refers to a nucleobase that has been determined or predicted for a nucleotide read. In particular, an observed nucleobase includes a nucleobase called by a sequencing device for a nucleotide read. For example, an observed nucleobase may comprise a called nucleobase that, after nucleotide reads have been mapped and aligned, aligns with a genomic coordinate corresponding to a cytosine base in a reference genome. In some implementations, the methylation-genotype-calling system can identify observed nucleobases at a given genomic coordinate from one or more nucleotide reads.

Also, as used herein, the term “target genomic sample” refers to a target genome or portion of a genome undergoing sequencing. For example, a genomic sample includes a sequence of nucleotides isolated or extracted from a sample organism (or a copy of such an isolated or extracted sequence). In particular, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device.

As further used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1: 1234570 or chr1: 1234570-1234870). Further, in certain implementations, a genomic coordinate refers to a source of a reference genome (e.g., mt for a mitochondrial DNA reference genome or SARS-COV-2 for a reference genome for the SARS-COV-2 virus) and a position of a nucleobase within the source for the reference genome (e.g., mt: 16568 or SARS-COV-2:29001). By contrast, in certain cases, a genomic coordinate refers to a position of a nucleobase within a reference genome without reference to a chromosome or source (e.g., 29727).

As used herein, the term “impute” (or “imputation”) refers to statistically inferring or estimating a genotype for a genomic coordinate or a genomic region. More specifically, imputing can include statistically inferring a genotype for one or more alleles corresponding to haplotypes for a genomic region of a genomic sample. For example, imputing can refer to utilizing marker variants surrounding a genomic region to determine genotype probabilities for alleles corresponding to haplotypes for the genomic region. In one or more embodiments, the methylation-genotype-calling system utilizes reference panels from a haplotype database and a genotype imputation model (e.g., Hidden Markov-based model) to impute genotype probabilities as a basis for genotype calls.

As used herein, the term “genotype probability” refers to a likelihood, probability, or score that a genomic sample comprises a particular genotype at a genomic coordinate or genomic region. In particular, a genotype probability may comprise a numerical score or measurement indicating the likelihood of a particular genotype. For example, a genotype probability may comprise a numerical score between 0 and 1, where a higher score corresponds with a greater likelihood of a given genotype. Accordingly, in some cases, a genotype probability includes a likelihood between 0 and 1 of a homozygous reference genotype, a likelihood of a heterozygous variant genotype, or a likelihood of a homozygous variant genotype at one or more genomic coordinates. Relatedly, the term “prior genotype probability” refers to an estimated genotype probability before new data or information is collected and/or analyzed (e.g., a newly determined metric or newly observed event). For example, a prior genotype probability can refer to an estimated genotype probability prior to imputation and/or prior to accounting for estimated methylation-level values. The term “posterior genotype probability” refers to a genotype probability that accounts for or reflects new data or information (e.g., a newly determined metric or newly observed event). For instance, a posterior genotype probability can refer to an estimated genotype probability as a result of imputation and/or that accounts for estimated methylation-level values or other metrics.

As used herein, the term “base-call-quality metric” refers to a specific score or other measurement indicating an accuracy of a nucleotide-base call. In particular, a base-call-quality metric comprises a value indicating a likelihood that one or more predicted nucleotide-base calls for a genomic coordinate contain errors. For example, in certain implementations, a base-call-quality metric can comprise a Q score (e.g., a Phred quality score) predicting the error probability of any given nucleotide-base call. To illustrate, a quality score (or Q score) may indicate that a probability of an incorrect nucleobase call at a genomic coordinate is equal to 1 in 100 for a Q20 score, 1 in 1,000 for a Q30 score, 1 in 10,000 for a Q40 score, etc.

As also used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequence determined as representative of an organism. For example, a linear human reference genome may be GRCh38 (or other versions of reference genomes) from the Genome Reference Consortium. GRCh38 may include alternate contiguous sequences representing alternate haplotypes or alternate nucleobases, such as SNPs and small indels (e.g., 10 or fewer base pairs, 50 or fewer base pairs).

As further used herein, the term “genotype call” refers to a determination or prediction of a particular genotype of a genomic sample at a genomic locus. In particular, a genotype call can include a prediction of a particular genotype of a genomic sample with respect to a reference genome or a reference sequence at a genomic coordinate or a genomic region. For instance, in some cases, a genotype call includes a determination or prediction that a genomic sample comprises both a nucleobase and a complementary nucleobase at a genomic coordinate that is either homozygous or heterozygous for a reference base or a variant (e.g., homozygous reference bases represented as 0|0 or heterozygous for a variant on a particular strand represented as 0|1). A genotype call is often determined for a genomic coordinate or genomic region at which a single nucleotide variant (SNV) or other variant has been identified for a population of organisms. In this disclosure, among other genotype calls, the methylation-genotype-calling system predicts genotype calls for SNV regions within a genomic sample.

As used herein, the term “plus strand” (or “positive strand”) refers to a strand of DNA in which the sequence corresponds directly to the sequence of an RNA transcript which is translated or translatable into a sequence of amino acids. Relatedly, the term “minus strand” (or “negative strand”) refers to an individual DNA strand that is complementary to the plus strand.

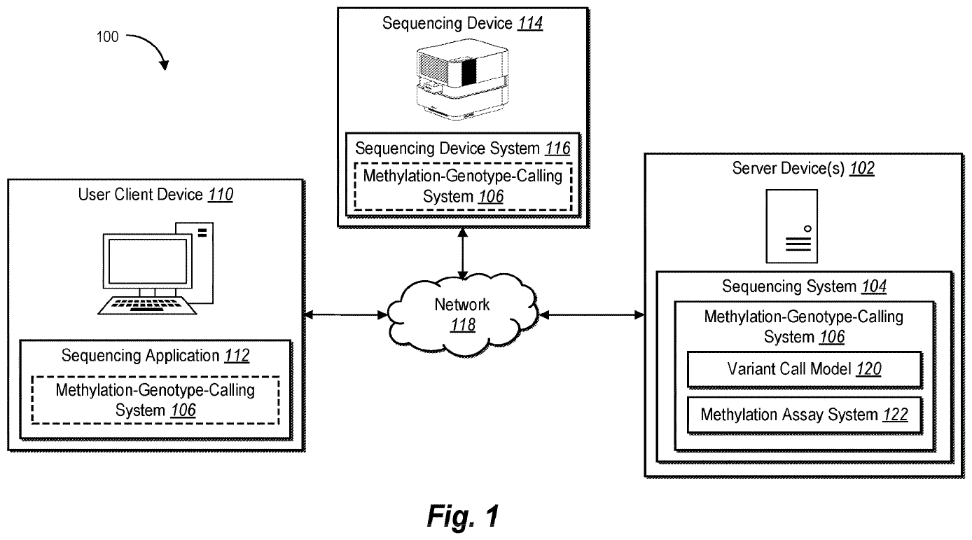

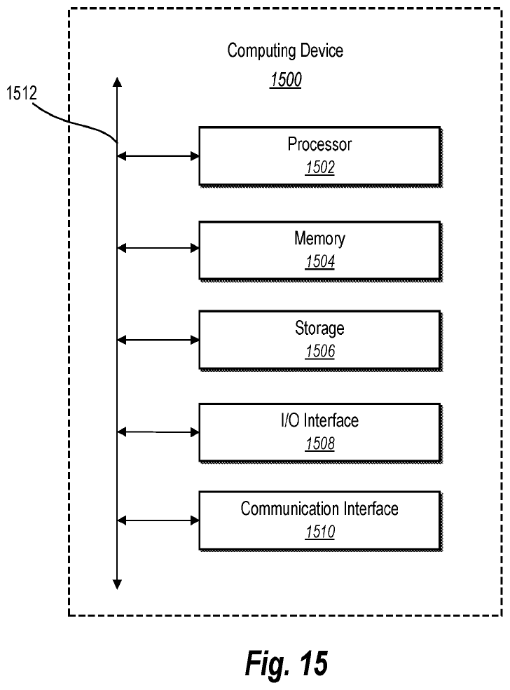

The following paragraphs describe a methylation-genotype-calling system with respect to illustrative figures that portray example embodiments and implementations. For example, FIG. 1 illustrates a schematic diagram of a computing system 100 in which a methylation-genotype-calling system 106 operates in accordance with one or more embodiments of the present disclosure. As illustrated, the computing system 100 includes server device(s) 102, a sequencing device 114, and a user client device 110 connected via a network 118. While FIG. 1 shows an embodiment of the methylation-genotype-calling system 106, this disclosure describes alternative embodiments and configurations below. As shown in FIG. 1, the sequencing device 114, the server device(s) 102, and the user client device 110 can communicate with each other via the network 118. The network 118 comprises any suitable network over which computing devices can communicate. Example networks are discussed in additional detail below with respect to FIG. 15.

As indicated by FIG. 1, the sequencing device 114 comprises a sequencing device system 116 for sequencing a genomic sample or other nucleic-acid polymer, such as when sequencing oligonucleotides extracted from a genomic sample as part of a methylation sequencing assay. In some embodiments, by executing the sequencing device system 116, the sequencing device 114 analyzes nucleotide sequences or oligonucleotides extracted from genomic samples to generate nucleotide reads or other data utilizing computer implemented methods and systems (described herein) either directly or indirectly on the sequencing device 114. More particularly, the sequencing device 114 receives nucleotide-sample slides (e.g., flow cells) comprising nucleotide sequences extracted from samples and subsequently copies and determines the nucleobase sequence of such extracted nucleotide sequences. As part of a methylation sequencing assay, for instance, the sequencing device 114 may determine nucleobase calls for nucleotide reads comprising CpG sites or other cytosine sites.

As suggested above, by executing the sequencing device system 116, the sequencing device 114 can run one or more sequencing cycles as part of a sequencing run. By executing the methylation-genotype-calling system 106, for instance, the sequencing device 114 can (i) sequence certain uracil bases that were converted from methylated, or unmethylated, cytosine bases and that are part of a nucleotide read and (ii) determine nucleobase calls of thymine for such uracil bases as part of a methylation sequencing assay. In one or more embodiments, the sequencing device 114 utilizes Sequencing by Synthesis (SBS) to sequence nucleic-acid polymers into nucleotide reads.

In some cases, the server device(s) 102 is located at or near a same physical location of the sequencing device 114 or remotely from the sequencing device 114. Indeed, in some embodiments, the server device(s) 102 and the sequencing device 114 are integrated into a same computing device. The server device(s) 102 may run a sequencing system 104 and/or the methylation-genotype-calling system 106 to generate, receive, analyze, store, and transmit digital data, such as by receiving base-call data, methylation assay data, and/or generating genotype calls.

As further suggested by FIG. 1, the sequencing device 114 may send (and the server device(s) 102 may receive) base-call data generated during a sequencing run of the sequencing device 114. By executing software in the form of the sequencing system 104 or the methylation-genotype-calling system 106, the server device(s) 102 may analyze read data and/or call data, such as sequencing metrics received from the sequencing device 114 and can determine a nucleobase sequence for a nucleotide read. In some embodiments, the sequencing system 104 of the server device(s) 102 determines the sequences of nucleobases in DNA and/or RNA segments or oligonucleotides. In addition to processing and determining sequences for nucleic acid polymers, the sequencing system 104 also generates a variant call file indicating one or more genotype calls and/or variant calls for one or more genomic coordinates. The server device(s) 102 may also communicate with the user client device 110. In particular, the server device(s) 102 can send data to the user client device 110, including a variant call file (VCF), or other information indicating nucleobase calls, methylation-level values, sequencing metrics, error data, or other metrics.

In some embodiments, the server device(s) 102 comprise a distributed collection of servers where the server device(s) 102 include a number of server devices distributed across the network 118 and located in the same or different physical locations. Further, the server device(s) 102 can comprise a content server, an application server, a communication server, a web-hosting server, or another type of server.

As further illustrated and indicated in FIG. 1, the user client device 110 can generate, store, receive, and send digital data. In particular, the user client device 110 can receive variant calls, methylation-level values, and corresponding sequencing metrics from the server device(s) 102 or receive base-call data (e.g., BCL or FASTQ) and corresponding sequencing metrics from the sequencing device 114. Furthermore, the user client device 110 may communicate with the server device(s) 102 to receive a VCF comprising nucleobase calls and/or other metrics, such as base-call-quality metrics or pass-filter metrics. The user client device 110 can accordingly present or display information pertaining to variant calls or other nucleobase calls within a graphical user interface to a user associated with the user client device 110. In particular, the user client device 110 can present results from a methylation sequencing assay or graphics that indicate methylation-level values for target cytosine bases.

Although FIG. 1 depicts the user client device 110 as a desktop or laptop computer, the user client device 110 may comprise various types of client devices. For example, in some embodiments, the user client device 110 includes non-mobile devices, such as desktop computers or servers, or other types of client devices. In yet other embodiments, the user client device 110 includes mobile devices, such as laptops, tablets, mobile telephones, or smartphones. Additional details regarding the user client device 110 are discussed below with respect to FIG. 15.

As further illustrated in FIG. 1, the user client device 110 includes a sequencing application 112. The sequencing application 112 may be a web application or a native application stored and executed on the user client device 110 (e.g., a mobile application, desktop application). The sequencing application 112 can include instructions that (when executed) cause the user client device 110 to receive data from the methylation-genotype-calling system 106 and present, for display at the user client device 110, base-call data (e.g., from a BCL), data from a VCF, or data from a methylation sequencing assay.

As further illustrated in FIG. 1, a version of the methylation-genotype-calling system 106 may be located on the user client device 110 as part of the sequencing application 112 or on the sequencing device 114 as part of the sequencing device system 116. In some embodiments, the methylation-genotype-calling system 106 is implemented by (e.g., located entirely or in part) on the user client device 110. In yet other embodiments, the methylation-genotype-calling system 106 is implemented by one or more other components of the computing system 100, such as the sequencing device 114. In particular, the methylation-genotype-calling system 106 can be implemented in a variety of different ways across the sequencing device 114, the user client device 110, and the server device(s) 102. As illustrated in FIG. 1, the methylation-genotype-calling system 106 is implemented by (e.g., entirely or in part) the sequencing system 104 implemented by the server device(s) 102. In at least one example, the methylation-genotype-calling system 106 can be downloaded from the server device(s) 102 to the sequencing device 114 and/or the user client device 110 where all or part of the functionality of the methylation-genotype-calling system 106 is performed at each respective device within the computing system.

As further illustrated in FIG. 1, the methylation-genotype-calling system 106 may implement a variant call model 120 and a methylation assay system 122. By executing the variant call model 120, the methylation-genotype-calling system 106 may align nucleotide reads with a reference genome and determine variant calls based on the aligned nucleotide reads. In some implementations, the methylation-genotype-calling system 106 analyzes each nucleotide of each read to determine (or receives information indicating) where the nucleotide read “fits” in relation to a reference sequence—e.g., where the bases within the read align with bases in the reference genome. In some cases, the methylation-genotype-calling system 106 aligns many nucleotide reads at a single genomic coordinate, thus resulting in a read pileup. In certain embodiments, the methylation assay system 122 is also implemented by the methylation-genotype-calling system 106. The methylation assay system 122 determines methylation-level values for CpG sites or other cytosine sites. As previously mentioned, the variant call model 120 and/or the methylation assay system 122 may be implemented by the methylation-genotype-calling system 106.

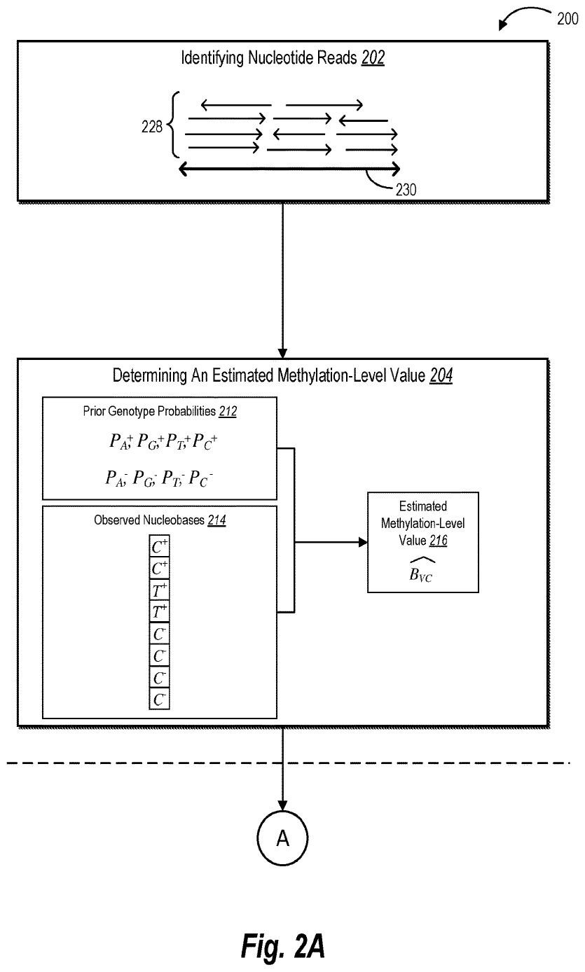

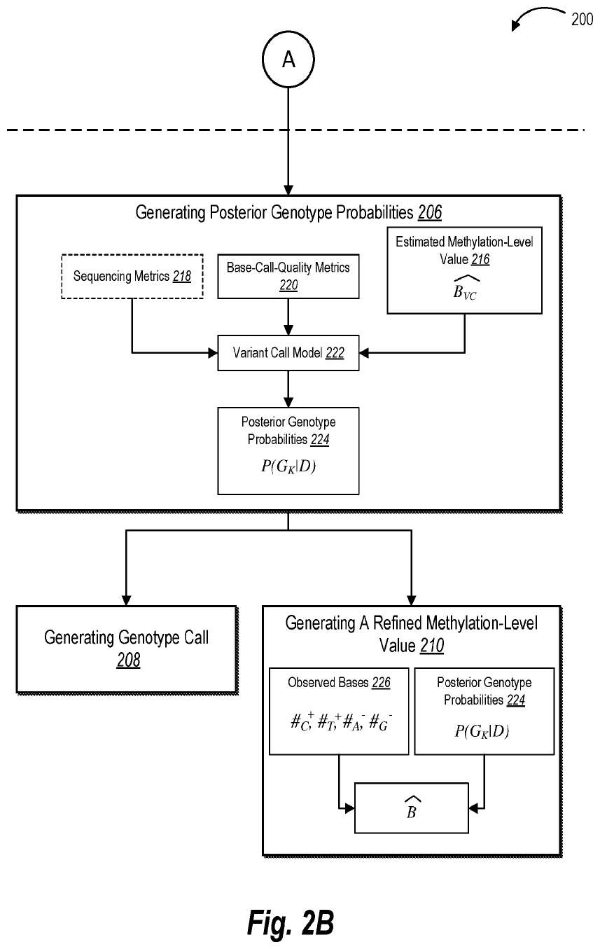

As mentioned previously, the methylation-genotype-calling system 106 may generate both genotype calls and methylation-level values based on nucleotide-read data. FIGS. 2A-2B illustrate an overview of the methylation-genotype-calling system 106 calling genotype and generating methylation-level values from a read pileup in accordance with one or more embodiments of the present disclosure. FIGS. 2A-2B illustrate a series of acts 200 comprising an act 202 of identifying nucleotide reads of a genomic sample, an act 204 of determining an estimated methylation-level value for nucleobase(s) at genomic coordinate(s), an act 206 of generating posterior genotype probabilities for the genomic sample at the genomic coordinate(s), an act 208 of generating a genotype call for the genomic sample at the genomic coordinate(s), and an act 210 of generating a refined methylation-level value for the nucleobase(s) at the genomic coordinate(s).

As shown in FIG. 2A, the methylation-genotype-calling system 106 performs the act 202 of identifying nucleotide reads. In particular, the methylation-genotype-calling system 106 performs an act of identifying, for a target genomic sample, nucleotide reads comprising one or more nucleobases converted by a methylation sequencing assay. In some cases, for instance, the methylation-genotype-calling system 106 receives data representing nucleotide reads 228 for a genomic sample that have been sequenced by a sequencing device. Such data for the nucleotide reads includes a sequence of nucleobase calls determined by a sequencing device. In some implementations, after receiving the read data, the methylation-genotype-calling system 106 aligns the nucleotide reads 228 with a reference genome 230. Based on the aligned nucleotide reads, the methylation-genotype-calling system 106 can determine one or more nucleobase calls for genomic coordinates and genomic regions of the target genomic sample with respect to the reference genome 230.

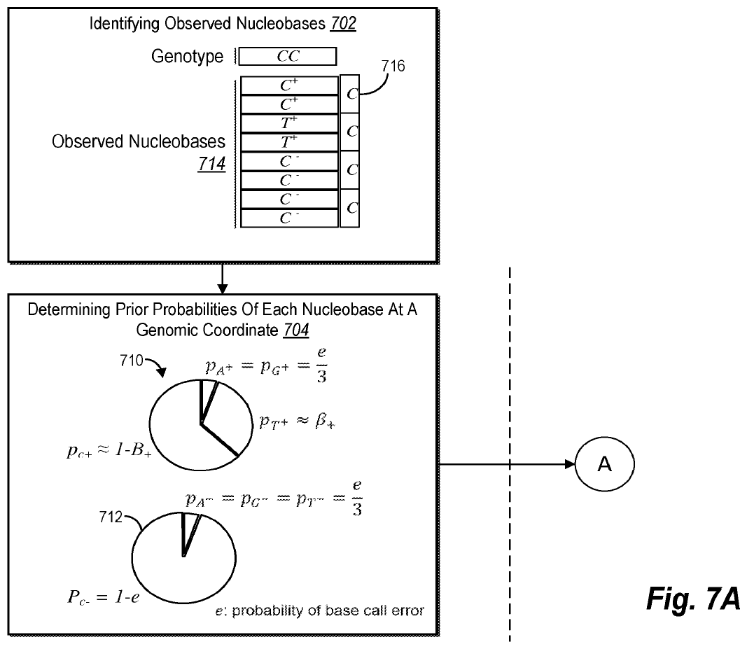

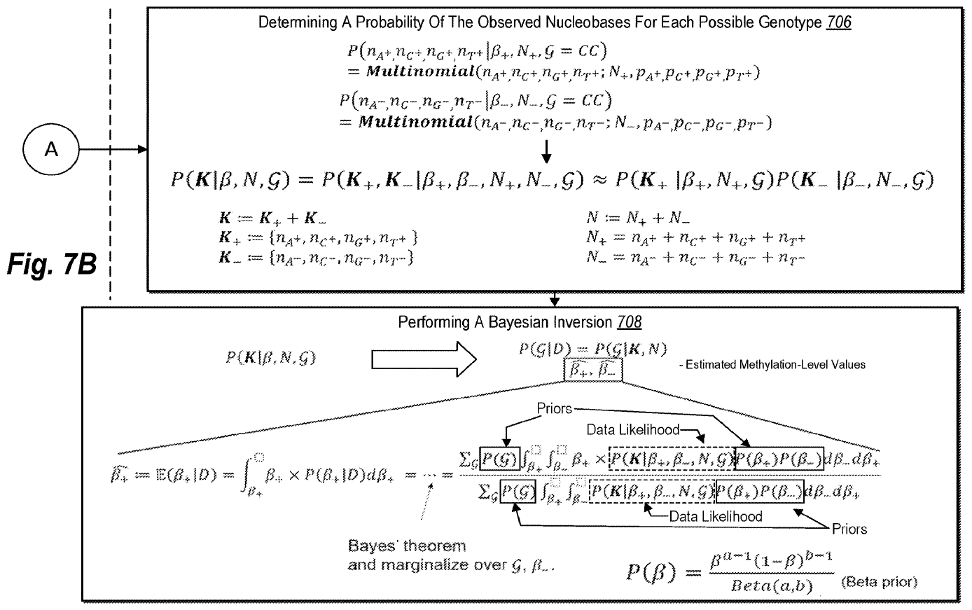

As further illustrated in FIG. 2A, the methylation-genotype-calling system 106 performs the act 204 of determining an estimated methylation-level value. In particular, the methylation-genotype-calling system 106 determines an estimated methylation-level value 216 ( [symbol] ) for a cytosine base (or other nucleobase) at a genomic coordinate based on prior genotype probabilities 212 corresponding to observed nucleobases 214 at the genomic coordinate within the nucleotide reads. As noted above, such estimated methylation-level values can be determined for genomic coordinates at which a genomic sample comprises a reference cytosine base or a nucleobase that could be called as cytosine (e.g., cytosine base on a minus strand). In some implementations, the methylation-genotype-calling system 106 utilizes a Bayesian method to determine the estimated methylation-level value. FIGS. 7A-7B illustrate the methylation-genotype-calling system 106 determining an estimated methylation-level value in accordance with one or more embodiments of the present disclosure.

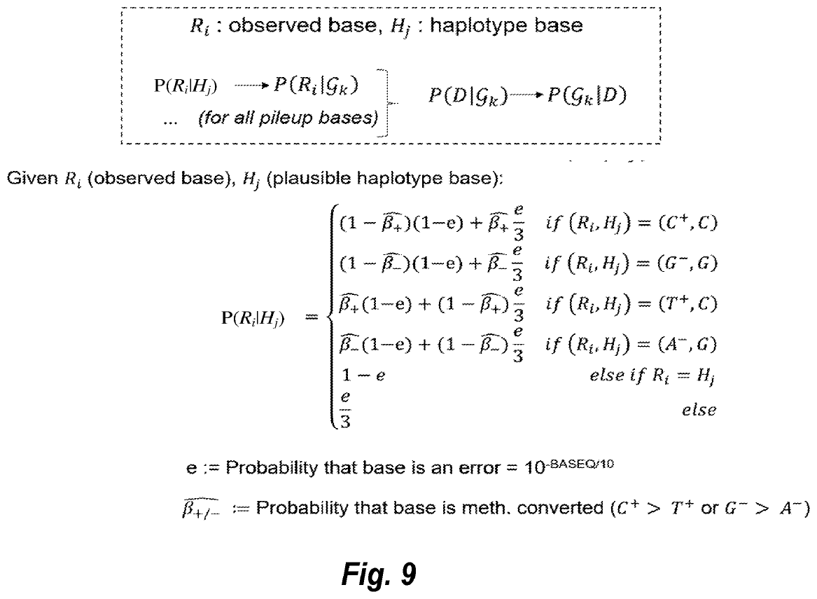

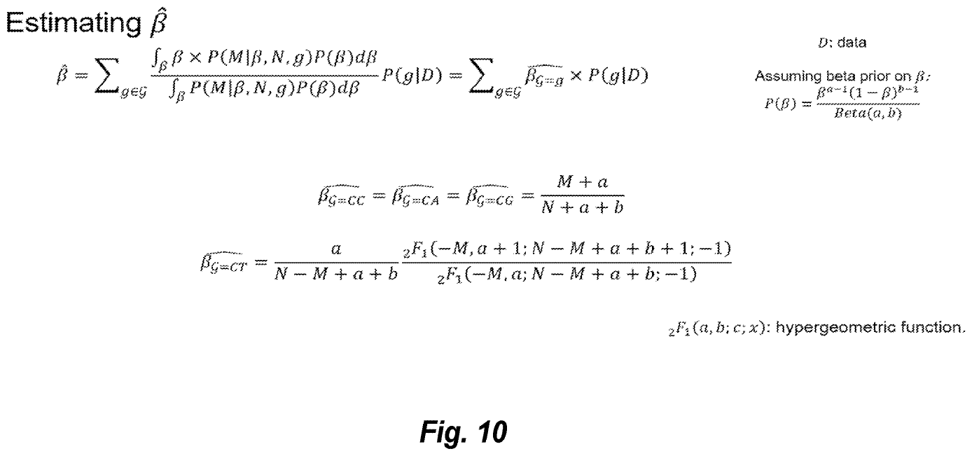

FIG. 2B illustrates the methylation-genotype-calling system 106 performing the act 206 of generating posterior genotype probabilities. In some implementations, the methylation-genotype-calling system 106 utilizes a variant call model 222 to generate posterior genotype probabilities 224 for the target genomic sample at the genomic coordinate based on an estimated methylation-level value 216 and base-call-quality metrics 220 for the observed nucleobases. In some implementations, the methylation-genotype-calling system 106 also utilizes sequencing metrics 218 (in addition to the base-call-quality metrics 220) as input into the variant call model 222 as part of generating the posterior genotype probabilities 224. FIGS. 9-10 and the corresponding paragraphs provide additional detail regarding how the methylation-genotype-calling system 106 generates posterior genotype probabilities in accordance with one or more embodiments of the present disclosure.

As further illustrated in FIG. 2B, the methylation-genotype-calling system 106 performs the act 208 of generating a genotype call. In some examples, based on the posterior genotype probabilities 224, the methylation-genotype-calling system 106 generates a genotype call for a target genomic sample at the genomic coordinate(s) corresponding to the observed nucleobases 214. For instance, the genotype call may indicate that the target genomic sample comprises a predicted combination of nucleobases at the genomic coordinate(s). In one or more implementations, the methylation-genotype-calling system 106 generates the genotype call for the target genomic sample by determining a predicted combination of nucleobases corresponding to a highest posterior genotype probability.

As further shown in FIG. 2B, in some implementations, the methylation-genotype-calling system 106 performs the act 210 of generating a refined methylation-level value. In particular, the methylation-genotype-calling system 106 may generate a refined methylation-level value (\hat{β}) for the cytosine nucleobase(s) at the genomic coordinate(s) based on the posterior genotype probabilities 224 and the observed nucleobases 226. Similar to the estimated methylation-level value 216, such refined methylation-level values can be determined for genomic coordinates at which a genomic sample comprises a reference cytosine base or a nucleobase that could be called as cytosine (e.g., cytosine base on a minus strand). The refined methylation-level value generated as part of the act 210 can be more accurate than the estimated methylation-level value 216. More specifically, in some instances, the methylation-genotype-calling system 106 determines the refined methylation-level value based on improved or updated input data—that is, the posterior genotype probabilities 224, a number of reads in the read pileup, and a number of methylated bases in the read pileup. FIG. 10 and the corresponding discussion further detail how the methylation-genotype-calling system 106 generates the refined methylation-level value in accordance with one or more implementations of the present disclosure.

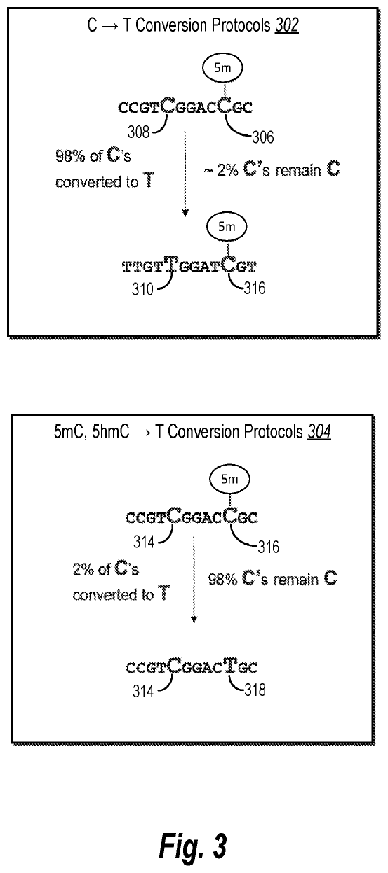

As mentioned, the methylation-genotype-calling system 106 identifies nucleotide reads comprising one or more nucleobases converted by a methylation sequencing assay. FIG. 3 illustrates various types of methylation sequencing protocols that the methylation-genotype-calling system 106 may utilize (or receive data from) as part of identifying nucleotide reads comprising converted nucleobases in accordance with one or more embodiments of the present disclosure. By way of overview, different methylation sequencing protocols utilize different conversions. For example, some methylation sequencing protocols convert unmethylated cytosine bases to thymine bases (i.e., C to T conversions). Other methylation sequencing protocols may convert methylated cytosine bases to thymine bases (i.e., 5mC, 5hmC to T conversions). The methylation-genotype-calling system 106 may utilize methylation data stemming from either type of methylation sequencing protocol. The following paragraphs further detail various effects of C to T conversion protocols 302 and 5mC, 5hmC to T conversion protocols 304, among other conversion protocols.

FIG. 3 illustrates the C to T conversion protocols 302. In the C to T conversion protocols 302, unmethylated bases are converted into thymine bases. For instance, the C to T conversion protocols 302 include Bisulfite and Enzymatic Methyl-seq (EM-seq) used for methylation sequencing assays. EM-seq can be performed, for instance, as described by Romualdas Vaisvila et al., Enzymatic Methyl Sequencing Detects DNA Methylation at Single-Base Resolution from Picograms of DNA, 30 Genome Research 1280-1289 (2021), which is hereby incorporated by reference in its entirety.

FIG. 3 depicts an example target genomic sample comprising an unmethylated cytosine base 308 and a methylated cytosine base 306. As illustrated in FIG. 3, the unmethylated cytosine base 308 is converted into a thymine base 310 while the methylated cytosine base 306 remains unconverted. As shown, 98% of cytosine bases are converted to thymine bases while about 2% of methylated cytosine bases remain cytosine bases.

FIG. 3 further illustrates 5mC, 5hmC to T conversion protocols 304. In these conversion protocols, methylated bases are converted into thymine bases. For instance, Tet-assisted pyridine borane sequencing (TAPS) uses a ten-eleven translocation (TET) enzyme for a methylation sequencing assay, as described by Yibin Liu et al., “Bisulfite-free Direct Detection of 5-Methylcystosine and 5-Hydroxymethylcystosine at Base Resolution,” 36 Nature Biotechnology 424-29 (2019). In some assays that rely on a TET enzyme, a methylation sequencing assay converts 5-Methylcystosine (5mC) and 5-Hydroxymethylcystosine (5hmC) into oxidized products using a TET enzyme and then uses an Apolipoprotein B mRNA Editing Enzyme, Catalytic Polypeptide (APOBEC) 3A or another APOBEC protein to deaminate unmodified cytosines by converting them to uracil bases. In some cases, such converted uracil bases are detected as thymine bases during sequencing.

As further shown in FIG. 3, the example target genomic sample comprises an unmethylated cytosine base 314 and a methylated cytosine base 316. As part of the 5mC, 5hmC to T conversion protocols 304, the methylated cytosine base 316 is converted to a thymine base 318, and the unmethylated cytosine base 314 remains unchanged. In contrast to the C to T conversion protocols 302, where the majority of cytosine bases are converted to thymine bases, only 2% of cytosine bases are converted to thymine bases in the 5mC, 5hmC to T conversion protocols 304. Subsequently, 98% of cytosine bases remain as cytosine bases. The methylation-genotype-calling system 106 can accurately and simultaneously generate methylation-level values and genotype calls for genomic samples that have undergone either C to T conversion protocols 302 or 5mC, 5hmC to T conversion protocols 304.

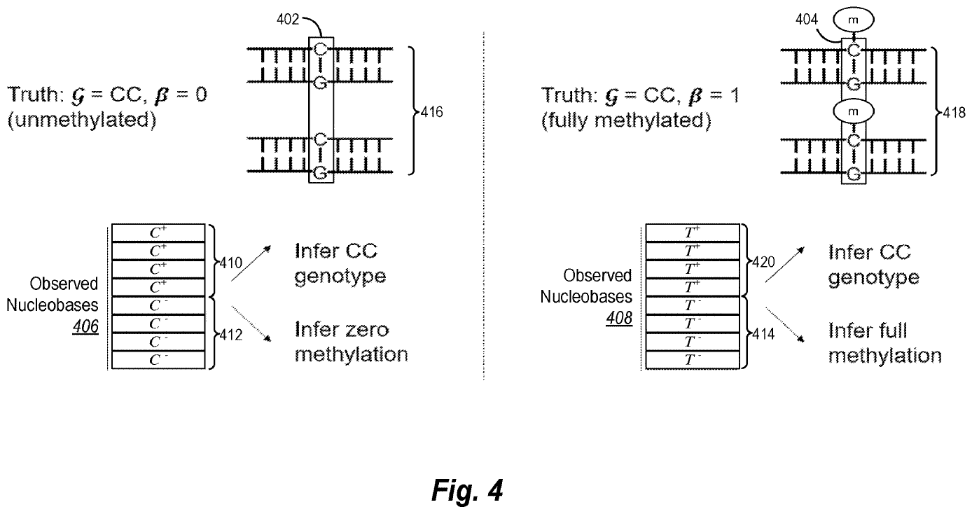

As mentioned previously, the methylation-genotype-calling system 106 estimates genotype and methylation-level values based on observed nucleobases. FIG. 4 illustrates the goal of the methylation-genotype-calling system 106 to accurately estimate genotype and methylation-level values based on observed nucleobases in accordance with one or more embodiments of the present disclosure. Generally, FIG. 4 illustrates a side-by-side comparison of two example genomic samples. More particularly, FIG. 4 includes (i) a genomic sample 416 comprising a fully unmethylated CC genotype and (ii) a genomic sample 418 comprising a fully methylated CC genotype. The genomic sample 416 and the genomic sample 418 include two homologous strands each—that is, each sample comprising nucleobases within a plus strand and a minus strand at a genomic coordinate. As indicated by FIG. 4, the goal of the methylation-genotype-calling system 106 is to accurately estimate the genotype and methylation-level value for a genomic coordinate.

As shown in FIG. 4, the methylation-genotype-calling system 106 aims to accurately estimate genotype and methylation-level values based on observed nucleobases. Generally, the methylation-genotype-calling system 106 accesses nucleotide read data to identify observed nucleobases at a particular genomic location. The observed nucleobases covering or overlapping a single genomic coordinate or region can also be referred to as a read pileup. To illustrate, observed nucleobases 406 represent multiple nucleotide reads corresponding to the genomic coordinate 402. The observed nucleobases 406 comprise observed nucleobases from four nucleotide reads for a plus strand 410 and observed nucleobases from four nucleotide reads for a minus strand 412. Similarly, observed nucleobases 408 comprise observed nucleobases from a plus strand 420 and observed nucleobases from a minus strand 414 at a genomic coordinate 404. FIG. 4 depicts such observed nucleobases at only a single genomic coordinate but not the remaining nucleobases from the corresponding nucleotide reads.

FIG. 4 illustrates the genomic sample 416 having cytosine bases on both haplotypes of the same strand (e.g., + strand) at a genomic coordinate 402. The genotype ( [symbol] ) at the genomic coordinate 402 for the genomic sample 416 is CC. More particularly, CC genotype represents part of the two haplotypes for the genomic sample 416 at the genomic coordinate 402, thereby illustrating that the genomic sample 416 is diploid. As further shown in FIG. 4, the cytosine bases at the genomic coordinate 402 are unmethylated (i.e., β=0). As described further below, the methylation-genotype-calling system 106 comprises instructions or an algorithm designed to accurately infer the CC genotype and zero methylation based on the observed nucleobases 406 from nucleotide reads that cover the genomic coordinate 402.

FIG. 4 also illustrates a genomic sample 418 having cytosine bases on both haplotypes of the same strand (e.g., + strand) at a genomic coordinate 404. The genotype ( [symbol] ) at the genomic coordinate 404 is CC. As shown, each of the cytosine bases at the genomic coordinate 404 are methylated (i.e., β=1). As described further below, the methylation-genotype-calling system 106 comprises instructions or an algorithm designed to accurately infer the CC genotype and full methylation based on the observed nucleobases 408 at the genomic coordinate 404.

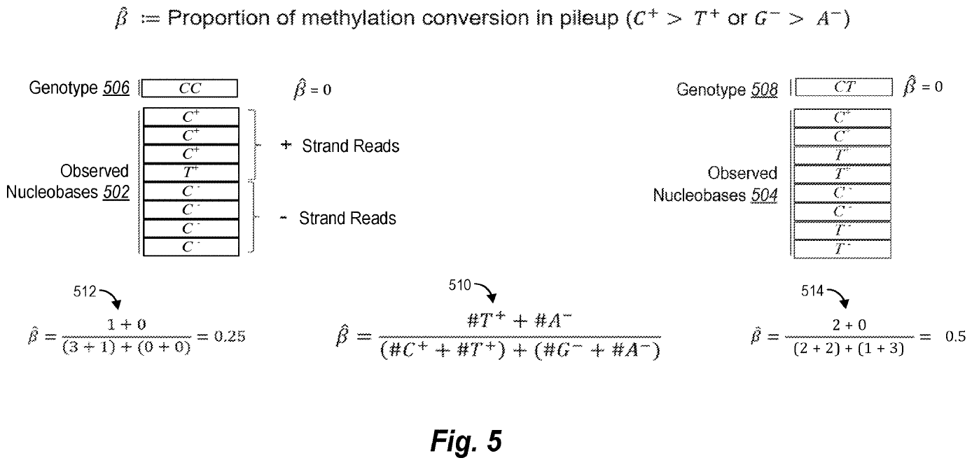

As mentioned previously, existing sequencing and methylation detection systems often generate inaccurate methylation-level values. FIG. 5 illustrates a model employed by existing sequencing and methylation detection systems that inaccurately estimate methylation-level values. More particularly, FIG. 5 illustrates how one or more existing sequencing and methylation detection systems attempt to estimate a methylation-level value (\hat{β}) for a genomic coordinate based on observed nucleobases. For example, existing sequencing and methylation detection systems seek to estimate methylation-level value representing a proportion of methylation conversion in a read pileup. In some examples, the methylation-level value indicates proportions of cytosine bases converted to thymine bases on a plus strand (C⁺>T⁺) or guanine bases converted to adenine bases on a minus strand (G⁻>A⁻).

FIG. 5 illustrates two examples of observed nucleobases from nucleotide reads covering two genomic coordinates. As shown in FIG. 5, a genotype 506 CC has been called for a first genomic sample at a first genomic coordinate. The cytosine bases at the first genomic coordinate are fully unmethylated (i.e., \hat{β}=0). As shown, the observed nucleobases 502 corresponding to the first genomic coordinate comprise three cytosine bases and one thymine base on the plus strand, and four cytosine bases on the minus strand.

FIG. 5 also illustrates a genotype 508 that has been called for a second genomic sample at a second genomic coordinate. As shown in FIG. 5, observed nucleobases 504 correspond to the genotype 508. The plus strand of the observed nucleobases 504 comprises two cytosine and two thymine bases. Similarly, the minus strand of the observed nucleobases 504 comprises two cytosine and two thymine bases. As further illustrated in FIG. 5, the genotype 508 of CT is fully unmethylated (i.e., \hat{β}=0).

Given the called genotypes 506 and 508 and the observed nucleobases 502 and 504, as shown in FIG. 5, existing sequencing and methylation detection systems often inaccurately estimate methylation-level values. In some examples, existing sequencing and methylation detection systems utilize the observed numbers of each nucleobase to estimate the methylation-level value (\hat{β}). For example, and shown in FIG. 5, existing sequencing and methylation detection systems may rely on equations such as equation 510 to estimate methylation levels. FIG. 5 illustrates equation implementation 512 and equation implementation 514 that demonstrate implementations of the equation 510 using different observed nucleobases. More specifically, the equation implementation 512 demonstrates an implementation of the equation 510 to the observed nucleobases 502, and the equation implementation 514 demonstrates an implementation of the equation 510 to the observed nucleobases 504. As shown by equation implementation 512, for the first genomic sample at the first genomic coordinate corresponding with the genotype 506, existing sequencing and methylation detection systems may estimate a methylation-level value of 0.25 based on the observed nucleobases 502. But the estimated methylation-level value of 0.25 diverges from the true methylation-level value of 0.

In some scenarios, existing sequencing and methylation detection systems estimate methylation-level values that are significantly off relative to the true methylation-level value. For instance, and as illustrated in FIG. 5, an existing sequencing and methylation detection system may utilize equation implementation 514 to estimate a methylation-level value for a cytosine base of the second genomic sample located at the second genomic coordinate corresponding with the genotype 508. The existing sequencing and methylation detection system vastly overestimates a methylation-level value of 0.5 when compared with the true methylation-level value of 0. As described below, the methylation-genotype-calling system 106 executes a model that avoids the inaccurate methylation-level-value estimation that results from the equation 510 employed by some existing sequencing and methylation detection systems.

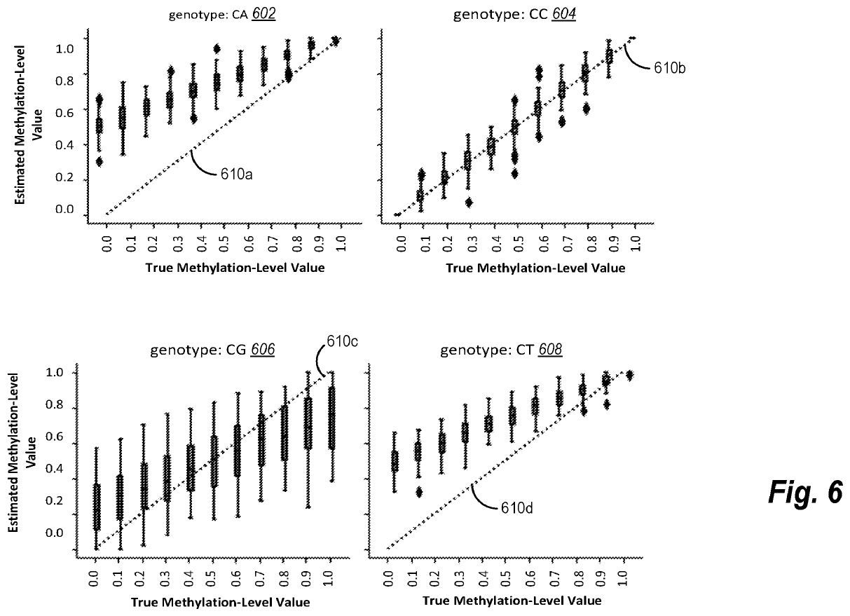

Technical deficiencies of existing sequencing and methylation detection systems often cause them to overestimate methylation-level values for particular genotypes. FIG. 6 and the corresponding paragraphs illustrate overestimated methylation-level values generated by existing sequencing and methylation detection systems. FIG. 6 illustrates graphs demonstrating estimated methylation-level values by existing sequencing and methylation detection systems compared with true methylation-level values for particular genotypes. By way of overview, FIG. 6 includes a graph 602 for the genotype CA, a graph 604 for the genotype CC, a graph 606 for the genotype CG, and a graph 608 for the genotype CT. The graphs illustrated in FIG. 6 also include reference lines 610a, 610b, 610c, and 610d that indicate estimated methylation-level values that would accurately equal true methylation-level values. While FIG. 6 illustrates graphs generated from simulated data, existing sequencing and methylation detection systems that employ the equation 510 would necessarily generate the estimated methylation-level values in the graphs of FIG. 6 using the simulated observed nucleobases.

As just indicated, the graphs illustrated in FIG. 6 portray results from simulated data where observed nucleotide reads are simulated. For example, a read pileup is simulated for each of the true genotypes (e.g., CA, CC, CG, and CT) and their true methylation-level values. Existing sequencing and methylation detection systems were utilized to analyze the simulated nucleotide reads and estimate methylation-level values.

As illustrated in FIG. 6, existing sequencing and methylation detection systems often overestimate methylation-level values for genotypes including adenine and thymine bases. More particularly, existing sequencing and methylation detection systems often assume that adenine and thymine bases are present because of methylation conversions. However, at genomic coordinates with true CA or CT genotypes depicted in FIG. 6, not all adenine bases or thymine bases represent methylation conversions. As shown by FIG. 6, existing sequencing and methylation detection systems often overestimate methylation-level values in the case of CA and CT genotypes. For example, the graph 602 and the graph 608 portray how the estimated methylation-level values are higher than the true methylation-level values.

As further shown in FIG. 6, existing sequencing and methylation detection systems can both overestimate and underestimate methylation-level values for CG genotypes. For CG genotypes, methylation can occur on both the plus strand and the minus strand. For example, a C on a plus strand can be methylated, and Cs on the minus strand that correspond with a G can also be methylated. Methylation of cytosine bases on both the plus and minus strands further corrupts the data that leads existing sequencing and methylation detection systems to both overestimate and underestimate methylation-level values for CG genotypes. As also depicted by the graph 606 in FIG. 6, existing sequencing and methylation detection systems often overestimate low-range methylation-level values and underestimate high-range methylation-level values for CG genotypes. For example, the graph 606 indicates that the existing sequencing and methylation detection system overestimates estimated methylation-level values on the left side of the graph 606 while underestimating methylation-level values relative to the reference line 610c.

As further shown in FIG. 6, in contrast to the other genotypes, existing sequencing and methylation detection systems more accurately estimate methylation-level values for CC genotypes. As illustrated in FIG. 6, the estimated methylation-level values shown in the graph 604 roughly tracks the reference line 610b. But such accuracy is limited to a true CC genotype.

As mentioned, the methylation-genotype-calling system 106 generates more accurate methylation-level estimates while calling genotypes for a genomic sample in part by leveraging prior genotype probabilities to determine an estimated methylation-level value for a cytosine base at a genomic coordinate. FIGS. 7A-7B illustrate a series of acts by which the methylation-genotype-calling system 106 determines an estimated methylation-level value for a cytosine base (or other candidate nucleobases) in accordance with one or more embodiments of the present disclosure. Generally, the methylation-genotype-calling system 106 utilizes a Bayesian method to determine the estimated methylation-level value for cytosine bases or a nucleobase that could be called as cytosine (e.g., cytosine base on a minus strand). By way of overview, FIGS. 7A-7B illustrate a series of acts comprising an act 702 of identifying observed nucleobases, an act 704 of determining prior probabilities of each nucleobase at a genomic coordinate, an act 706 of determining a probability of the observed nucleobases for each possible genotype, and an act 708 of performing a Bayesian inversion.

As shown in FIG. 7A, the methylation-genotype-calling system 106 performs the act 702 of identifying observed nucleobases. Generally, the methylation-genotype-calling system 106 considers genotypes that can be methylated. In some implementations, the methylation-genotype-calling system 106 performs the act 704 by first determining a genomic coordinate of a cytosine base in the target genomic sample. Some existing sequencing and methylation detection systems determine the genomic coordinates of cytosine bases by referring to a reference genome. However, these existing systems may fail to evaluate cytosine bases in the target genomic sample that do not align with cytosine bases in the reference genome. Accordingly, in some implementations, the methylation-genotype-calling system 106 identifies genomic coordinates within the target genomic sample that could be called as having a cytosine. Thus, the methylation-genotype-calling system 106 predicts methylation levels for genomic coordinates containing a cytosine allele. More specifically, the methylation-genotype-calling system 106 identifies and considers (i) genomic coordinates where the reference base is cytosine base and the called genotype contains a cytosine base or (ii) genomic coordinates in the target genomic sample where the reference base is not a cytosine base (e.g., ref=A) but the called genotype contains a cytosine base. In some examples, the methylation-genotype-calling system 106 identifies genomic coordinates in the target genomic sample that could be called as a cytosine base based on prior genotype likelihoods.

Furthermore, and as part of performing the act 702 of identifying observed nucleobases, the methylation-genotype-calling system 106 identifies nucleotide reads corresponding to the genomic coordinate of the cytosine base in the target genomic sample. As described previously, the methylation-genotype-calling system 106 identifies nucleotide reads that cover or align with the genomic coordinate of a cytosine base in the reference genome. Additionally, or alternatively, the methylation-genotype-calling system 106 identifies nucleotide reads that cover or align with a genomic coordinate of a nucleobase in the target genomic sample that could be called as a cytosine. The methylation-genotype-calling system 106 can compile a read pileup comprising observed nucleobases 714 at the genomic coordinate within the nucleotide reads. For example, the observed nucleobases 714 align with a genomic coordinate having a cytosine base in the reference genome or the target genomic sample.

As further shown in FIG. 7A, the methylation-genotype-calling system 106 may determine observed nucleobases corresponding with a plus strand and a minus strand of the target genomic sample. For example, and as illustrated in FIG. 7A, the methylation-genotype-calling system 106 identifies the observed nucleobases aligning with a genomic coordinate having a true genotype ( [symbol] ) of CC. As shown as part of the act 702, the methylation-genotype-calling system 106 identifies two plus strand cytosine bases (C⁺) and two plus strand thymine bases (T⁺). The methylation-genotype-calling system 106 identifies four minus strand cytosine bases (C⁻). The plus strand nucleobases comprise nucleobase predictions for sample nucleobases 716 at the genomic coordinate.

As indicated by FIG. 7A, the combination of the observed nucleobases 714 illustrated in FIG. 7A provide evidence for a CT genotype or a methylated CC genotype. In particular, the plus strand thymine bases may be evidence of a converted methylated (or unmethylated) plus strand cytosine base. Accordingly, the methylation-genotype-calling system 106 accounts for both such genotypes in determining prior probabilities.

FIG. 7A further illustrates the methylation-genotype-calling system 106 performing the act 704 of determining prior probabilities of each nucleobase at a genomic coordinate. As mentioned previously, the methylation-genotype-calling system 106 determines prior probabilities of each nucleobase at a genomic coordinate comprising at least one cytosine base (e.g., CC, CT, CA, or CG). Generally, the methylation-genotype-calling system 106 assumes that genomic coordinates with observed nucleobases supporting genotypes or haplotypes without a cytosine base are not subject to methyl conversion and, therefore, does not utilize the Bayesian conversion depicted in FIGS. 7A-7B as part of determining genotype calls for such genomic coordinates. For genomic coordinates with observed nucleobases supporting a genotype with a cytosine base, however, the methylation-genotype-calling system 106 proceeds as depicted in FIGS. 7A-7B and predict prior probabilities of nucleobases and, subsequently, posterior genotype probabilities for such genomic coordinates by incorporating a methylation-level value.

As indicated by chart 710 in FIG. 7A, in some embodiments, the methylation-genotype-calling system 106 accesses or identifies prior probabilities for the observed nucleobases 714 on the plus strand. As an overview of the chart 710, the methylation-genotype-calling system 106 determines that (i) the prior probability of a thymine base on a plus strand is approximately equal to a beta value, where the beta value represents a position-specific fraction of cytosine bases methyl-converted to thymine bases, (ii) the prior probability of a cytosine base on the plus strand is approximately equal to the formula 1 minus the beta value, and (iii) the prior probability of an adenine or guanine base on the plus strand is determined by the base-call-error probability (e.g., a base-call error over 3).

As just indicated and as shown with more particularity in FIG. 7A, the chart 710 depicts prior probabilities of each nucleobase at a genomic coordinate on a plus strand. As shown, the methylation-genotype-calling system 106 assumes that prior probabilities of plus strand adenine bases and plus strand guanine bases are largely unaffected by methylation assay conversions. Accordingly, in some implementations, the methylation-genotype-calling system 106 determines that a prior probability of a plus strand adenine base (p_{A}_{⁺}) and the prior probability of a plus strand guanine base (p_{G}_{⁺}) equal-, where e represents a prior probability of base call error.

As mentioned, a plus strand thymine base (T⁺) may be evidence of a converted methylated (or unmethylated) plus strand cytosine base. More specifically, a prior probability of a plus strand thymine reflects a likelihood that a cytosine base has been converted to a thymine base. Accordingly, the prior probability of a plus strand thymine base (p_{T}_{⁺}) is approximately equal to a plus-strand-methylation-level value (β₊). Thus, the prior probability of a plus strand thymine base at a genomic coordinate can be expressed by the equation p_{T}_{⁺}≈β₊. In some implementations, the plus-strand-methylation-level value (β₊) represents a cytosine methylation percentage on the plus strand.

As further illustrated in FIG. 7A, the methylation-genotype-calling system 106 considers that plus strand cytosine bases (C⁺) can be methyl-converted to thymine bases. Thus, the methylation-genotype-calling system 106 determines that a prior probability of a plus strand cytosine base is not only dependent on base call error but whether the cytosine base has been methyl-converted into a thymine. Accordingly, the methylation-genotype-calling system 106 may determine that p_{C}_{⁺}≈1−β₊ where p_{C}_{⁺} represents the prior probability of a plus strand cytosine base at a genomic coordinate, and β₊ represents the plus-strand-methylation-level value.

In some embodiments, and as shown in FIG. 7A, the methylation-genotype-calling system 106 does not incorporate the prior probability of base call error (e) into the probability of a plus strand cytosine base (p_{C+}) or the probability of a plus strand cytosine base (p_{T}_{⁺}). More particularly, p_{C}_{⁺} and p_{T}_{⁺} are approximate, and in many instances, the prior probability of base call error is negligible in comparison to the plus-strand-methylation-level value (β₊) and 1−β₊. In some implementations, the methylation-genotype-calling system 106 incorporates the prior probability of base call error into p_{C}_{⁺} or p_{T}_{⁺}. For example, in some implementations, the methylation-genotype-calling system 106 approximates p_{T}_{⁺} to equal

$${β}_{+} + \frac{e}{3} .$$

The effects of methyl-conversions on the prior probabilities of plus strand nucleotide bases have analogous cases involving the complementary bases on the minus strand. As an overview of the chart 712, the methylation-genotype-calling system 106 determines that the prior probability of an adenine, guanine, or thymine base on the minus strand is likewise determined by the base-call-error probability. Further, the prior probability of cytosine on the plus strand is approximately equal to the formula 1 minus the base-call-error probability. In particular, the chart 712 depicts probabilities of each nucleobase at a genomic coordinate on a minus strand. In some implementations, the methylation-genotype-calling system 106 determines that the prior probability of a minus strand adenine base (p_{A}_{^{−}}), the prior probability of a minus strand guanine base (p_{G}_{^{−}}), and the prior probability of a minus strand thymine base (p_{T}_{^{−}}) equal e/3, where e represents the prior probability of a base call error. In some examples, the methylation-genotype-calling system 106 divides e by 3 because the methylation-genotype-calling system 106 assumes that an erroneous base call could be called as any one of a minus strand adenine base (A⁻), a minus strand guanine base (G⁻), or a minus strand thymine base (T⁻). The methylation-genotype-calling system 106 does not record which error is most likely and assumes that the probability of all three are equal. The methylation-genotype-calling system 106 applies analogous reasoning to determining p_{A}_{⁺}, p_{G}_{⁺}, and (in some implementations) p_{T}_{⁺} and utilizes the same e/3 value when determining impacts of base call error in those probabilities.

As further shown by the chart 712 in FIG. 7A, the methylation-genotype-calling system 106 determines that a prior probability of a minus strand cytosine base (p_{C}_{^{−}}) equals 1−e, where e represents the probability of a base call error. This reflects an assumption by the methylation-genotype-calling system 106 that, assuming an underlying CC genotype, the methylation-genotype-calling system 106 would observe cytosine bases at a higher probability. Under this assumption, the methylation-genotype-calling system 106 would predict lower probabilities of observing adenine, guanine, and thymine bases that are proportional to base calling error. Furthermore, a prior probability of a minus strand cytosine base is generally unaffected by methylation because a minus strand cytosine base (C⁻) cannot be methylated. C⁻ corresponds with a G on the original DNA template.

As shown in FIG. 7B, the methylation-genotype-calling system 106 performs the act 706 of determining a probability of the observed nucleobases for each possible genotype. In some embodiments, the methylation-genotype-calling system 106 determines the prior probabilities of the observed nucleobases at the genomic coordinate based on numbers of each observed nucleobase from nucleotide reads of a target genomic sample at a genomic coordinate, the probability of each nucleobase at the genomic coordinate, and corresponding prior genotype probabilities. For example, in some implementations, the methylation-genotype-calling system 106 determines the prior probabilities of observed nucleobases at the genomic coordinate on the plus strand utilizing the following equation:

$$P ( {n}_{{A}^{+}} , {n}_{{C}^{+}} , {n}_{{G}^{+}} , {n}_{{T}^{+}} \vert {β}_{+} , {N}_{+} , 𝒢 = CC ) = Multinomial ( {n}_{{A}^{+}} , {n}_{{C}^{+}} , {n}_{{G}^{+}} , {n}_{{T}^{+}} ; {N}_{+} , {p}_{{A}^{+}} , {p}_{{C}^{+}} , {p}_{{G}^{+}} , {p}_{{T}^{+}} )$$

where n_{A}_{⁺} represents a number of observed plus strand adenine bases, n_{C}_{⁺} represents a number of observed plus strand cytosine bases, n_{G}_{⁺} represents a number of observed plus strand guanine bases, and n_{T}_{⁺} represents a number of observed plus strand thymine bases at the genomic coordinate. In the equation above, β₊ represents the plus-strand-methylation-level value and N₊ represents a total number of observed nucleobases on the plus strand. For example, N₊=n_{A}_{⁺}+n_{C}_{⁺}+n_{G}_{⁺}+n_{T}_{⁺}. The methylation-genotype-calling system 106 determines the probabilities of the observed nucleobases on the plus strand given the hypothesis that a given genotype is the true underlying genotype. For example, and as shown in FIG. 7B, the methylation-genotype-calling system 106 determines the probabilities of the observed nucleobases given a true genotype of CC ( [symbol] =CC). In some embodiments, the methylation-genotype-calling system 106 determines the probabilities of the observed nucleobases at the genomic coordinate given each possible true genotype (e.g., AA, AC, AG, AT, CC, CG, CT, GG, GT, TT).

In some embodiments, as part of performing the act 706, the methylation-genotype-calling system 106 utilizes the following equation to determine the probabilities of observed nucleobases at the genomic coordinate on the minus strand:

$$P ( {n}_{{A}^{-} ,} {n}_{{C}^{-}} , {n}_{{G}^{-}} , {n}_{{T}^{-}} \vert {β}_{-} , {N}_{-} , 𝒢 = CC ) = Multinomial ( {n}_{{A}^{-}} , {n}_{{C}^{-}} , {n}_{{G}^{-}} , {n}_{{T}^{-}} ; {N}_{-} , {p}_{{A}^{-}} , {p}_{{C}^{-}} , {p}_{{G}^{-}} , {p}_{{T}^{-}} )$$

where n_{A}_{^{−}} represents a number of observed minus strand adenine bases, n_{C-} represents a number of observed minus strand cytosine bases, n_{G}_{^{−}} represents a number of observed minus strand guanine bases, and n_{T}_{^{−}} represents a number of observed minus strand thymine bases at the genomic coordinate. In the equation above, β₋ represents the minus-strand-methylation-level value (or a cytosine methylation percentage on the minus strand), and N₋ represents a total number of observed nucleobases on the minus strand. In some embodiments, N₋=n_{A}_{^{−}}+n_{C}_{^{−}}+n_{G}_{^{−}}+n_{T}_{^{−}}. The methylation-genotype-calling system 106 determines the probabilities of the observed nucleobases on the minus strand given the hypothesis that a given genotype is the true underlying genotype. For example, and as shown in FIG. 7B, the methylation-genotype-calling system 106 determines the probabilities of the observed nucleobases given a true genotype of CC ( [symbol] =CC). The methylation-genotype-calling system 106 determines probabilities of observed nucleobases for each possible underlying genotype.

As described above, the methylation-genotype-calling system 106 determines probabilities of observed nucleobases at the genomic coordinates for the plus strand and the minus strand. As further shown in FIG. 7B, the methylation-genotype-calling system 106 condenses the probabilities for plus and minus strands into a probability of the observed nucleobases (K) for the whole observed pileup at a given genomic coordinate. In some implementations, the methylation-genotype-calling system 106 determines the probability of the observed nucleobases at the given genomic coordinate using the following equation:

$$\begin{matrix}P ( K \vert β , N , 𝒢 ) = P ( {K}_{+} , {K}_{-} \vert {β}_{+} , {β}_{-} , {N}_{+} , {N}_{-} , 𝒢 ) \\ \approx P ( {K}_{+} \vert {β}_{+} , {N}_{+} , 𝒢 ) P ( {K}_{-} \vert {β}_{-} , {N}_{-} , 𝒢 )\end{matrix}$$

where K₊ represents observed nucleobases at the genomic coordinate for the plus strand, and K₋ represents observed nucleobases at the genomic coordinate for the minus strand. In the equation above, β represents a methylation value or a cytosine methylation percentage at the genomic coordinate, β₊ represents the plus-strand-methylation-level value, and β₋ represents the minus-strand-methylation-level value. N represents a total number of observed nucleobases at the genomic coordinate and equals a sum of the total number of observed nucleobases on the plus strand (N₊) and the total number of observed nucleobases on the minus strand (N₋). As mentioned, [symbol] represents a hypothesized true genotype, and the methylation-genotype-calling system 106 determines the probability of the observed nucleobases for each possible genotype at the genomic coordinate. The methylation-genotype-calling system 106 enumerates across all possible genotypes.

As further shown in FIG. 7B, the methylation-genotype-calling system 106 performs the act 708 of performing a Bayesian inversion. More specifically, the methylation-genotype-calling system 106 generates an estimated methylation-level value ( [symbol] ) by performing a Bayesian inversion on the probabilities of the observed nucleobases determined in the act 706, as represented by (P(K|β,N, [symbol] )). The Bayesian inversion can be expressed using the following equation:

$$P ( 𝒢 \vert D ) = ( P ( 𝒢 \vert K , N )$$

where the methylation-genotype-calling system 106 determines the prior genotype probability ( [symbol] ) for each potential genotype given the data (D). More specifically, the methylation-genotype-calling system 106 determines the prior genotype probabilities given the observed nucleobases at the genomic coordinate (K) and the total number of observed nucleobases at the genomic coordinate (N).

As further illustrated in FIG. 7B, the methylation-genotype-calling system 106 further performs the Bayesian inversion to solve for the plus-strand-methylation-level value ( [symbol] ) and the minus-strand-methylation-level value ( [symbol] ). As shown, the methylation-genotype-calling system 106 produces estimated methylation-level values for both the plus strand and the minus strand. In one example, in the case of a CG heterozygous position, the methylation-genotype-calling system 106 produces a plus-strand-methylation-level value ( [symbol] ) for the plus strand of the C haplotype and minus-strand-methylation-level value ( [symbol] ) for the minus strand of the G haplotype. By way of example, the methylation-genotype-calling system 106 may determine the plus-strand-methylation-level value using the following equation:

$$\begin{matrix}\text{?} := 𝔼 ( {β}_{+} \vert D ) = {\int }_{β}^{} {β}_{+} \times P ( {β}_{+} \vert D ) d {β}_{+} = \text{} ⋯ \\ = \frac{{\sum }_{𝒢} P ( 𝒢 ) {\int }_{{β}_{+}}^{} {\int }_{{β}_{-}}^{} {β}_{+} \times P ( K \vert {β}_{+} , {β}_{-} , N , 𝒢 ) P ( {β}_{+} ) P ( {β}_{-} ) d {β}_{-} d {β}_{+}}{{\sum }_{𝒢} P ( 𝒢 ) {\int }_{{β}_{+}}^{} {\int }_{{β}_{-}}^{} P ( K \vert {β}_{+} , {β}_{-} , N , 𝒢 ) P ( {β}_{+} ) P ( {β}_{-} ) d {β}_{-} d {β}_{+}}\end{matrix}$$

where the methylation-genotype-calling system 106 estimates an expected value ( [symbol] ) of a plus-strand-methylation-level value (β₊) given data (D). In some implementations, the methylation-genotype-calling system 106 estimates β₊ by integrating over all possible values of β₊ from 0 to 1. Generally, the methylation-genotype-calling system 106 solves the integrals analytically to arrive at the above expression, which comprises a single fraction of the sum of different terms—each being a genotype-specific term. As shown in the equation above, P( [symbol] ) represents prior genotype probabilities and P(K|β₊,β₋,N, [symbol] ) represents the probabilities of the observed nucleobases at the genomic coordinate. The above fraction also includes a probability of a plus-strand-methylation-level value P(β₊) and a probability of a minus-strand-methylation-level value P(β₋). These probabilities can be tuned using a beta prior function expressed below

$$P ( β ) = \frac{{{β}^{a - 1} ( 1 - β )}^{b - 1}}{Beta ( a , b )} ( Beta Prior )$$

where a and b represent tuneable parameters. When a and b are equal to one, the distribution collapses to a uniform distribution. In some implementations, the methylation-genotype-calling system 106 tunes a and b to find the beta prior that yields the highest accuracy.

In some implementations, the methylation-genotype-calling system 106 generates the estimated methylation-level value ( [symbol] ) for a genomic coordinate by combining the plus-strand-methylation-level value ( [symbol] ) and the minus-strand-methylation-level value ( [symbol] ). The methylation-genotype-calling system 106 may utilize the estimated methylation-level value to improve the accuracy of genotype calling. Furthermore, the methylation-genotype-calling system 106 may refine the estimated methylation-level value ( [symbol] ) to generate a refined methylation-level value (\hat{β}). FIG. 10 and the corresponding paragraphs illustrate an embodiment of the methylation-genotype-calling system 106 generating a refined methylation-level value in accordance with one or more embodiments.

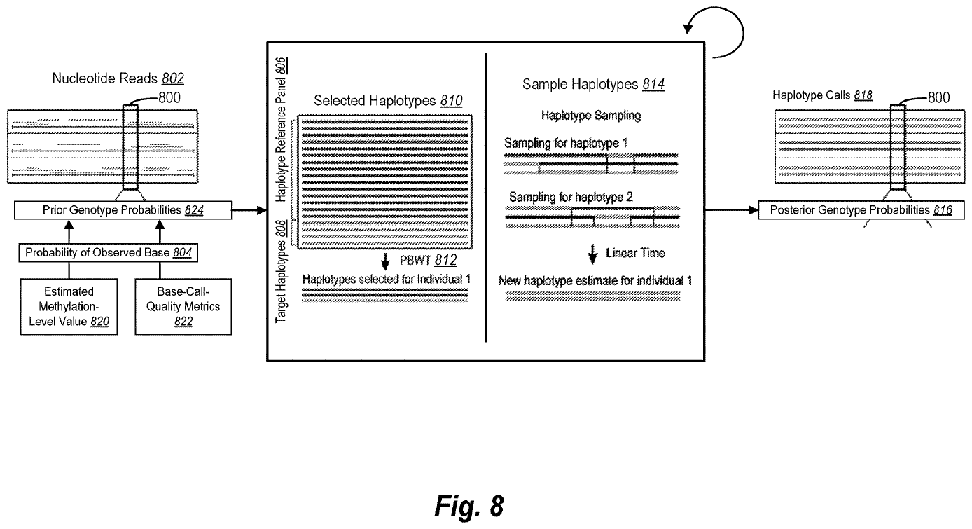

As mentioned, in some embodiments, the methylation-genotype-calling system 106 utilizes a variant call model to generate posterior genotype probabilities based on the estimated methylation-level value and base-call quality metrics for the observed nucleobases. In some cases, the variant call model includes an imputation model, among other components. FIG. 8 illustrates an overview of the methylation-genotype-calling system 106 generating posterior genotype probabilities in accordance with one or more embodiments of the present disclosure. In some examples, the methylation-genotype-calling system 106 applies a genotype imputation model, such as a Hidden Markov Model (HMM)-based genotype imputation model, to nucleotide reads corresponding to a genomic coordinate. By applying the genotype imputation model, the methylation-genotype-calling system 106 can determine posterior genotype probabilities 816 and haplotype calls 818 for the genomic region. FIG. 8 illustrates the methylation-genotype-calling system 106 applying a Genotype Likelihoods Imputation and PhaSing mEthod (GLIMPSE) as a genotype imputation model to determine the posterior genotype probabilities 816 at a genomic coordinate.

Through imputation, the methylation-genotype-calling system 106 imputes one or more genotype calls for a target genomic sample. As shown in FIG. 8, for instance, the methylation-genotype-calling system 106 determines a probability of an observed base 804 for a genomic coordinate 800 from a target genomic sample. The probability of an observed base 804 is expressed as P(Rᵢ|Hⱼ) where Rᵢ represents a given observed nucleobase, and Hⱼ represents the base of the candidate haplotype being considered. For example, the methylation-genotype-calling system 106 considers the observed nucleobases and tests them against candidate haplotypes that have been identified. The methylation-genotype-calling system 106 attempts to align the observed nucleobase with the haplotype in various ways to identify possible combinations. For example, if the nucleotide reads 802 comprise only adenine or cytosine bases at the genomic coordinate 800, the methylation-genotype-calling system 106 tests to determine whether the underlying haplotype is an adenine base or a cytosine base.

In some implementations, the methylation-genotype-calling system 106 determines the probability of an observed base 804 based on an estimated methylation-level value 820 ( [symbol] ) and base-call-quality metrics 822. Each of the base-call-quality metrics 822 indicate a probability that an observed base in a nucleotide read is an error. More specifically, the methylation-genotype-calling system 106 determines the observed base 804 based on a base-call-quality metric (e.g., a BASEQ score) for the observed base. The estimated methylation-level value 820 indicates a probability that an observed base has been converted by a methylation sequencing assay. FIG. 9 and the corresponding paragraphs provide additional detail regarding how the methylation-genotype-calling system 106 determines the probability of an observed base 804 in accordance with one or more embodiments.

As shown in FIG. 8, the methylation-genotype-calling system 106 generates prior genotype probabilities 824 based on the observed base 804. In particular, the methylation-genotype-calling system 106 can aggregate probabilities of observed bases for all observed pileup bases P(Rᵢ|Hⱼ) to generate probabilities of observed bases for candidate genotypes. The probabilities of observed bases for candidate genotypes can be expressed as P(Rᵢ| [symbol] ), where Rᵢ represents a given observed nucleobase, and [symbol] represents the bases of the candidate genotype being considered. In some implementations, the methylation-genotype-calling system 106 generates the prior genotype probabilities 824 based on the observed bases for candidate genotypes. For example, in some implementations, the methylation-genotype-calling system 106 generates the prior genotype probabilities 824 by multiplying the probabilities of observed bases to determine corresponding candidate genotypes.

As further indicated by FIG. 8, the genomic coordinate 800 corresponds to variable positions (or variable genomic coordinates) of a haplotype reference panel 806. In certain cases, the methylation-genotype-calling system 106 further deconvolves a vector of the probability of an observed base 804 to two independent vectors of haplotype allele likelihoods (or, simply, haplotype likelihoods), where each vector corresponds to one of two complementary haplotypes.

Based on the haplotype likelihoods from the independent vectors, in some implementations, the methylation-genotype-calling system 106 imputes two target haplotypes as the haplotype calls 818 using a haploid version of an HMM in an iterative process. As shown in FIG. 8, for instance, the methylation-genotype-calling system 106 selects haplotypes 810 based on the haplotype reference panel 806 and target haplotypes 808 estimated for each genomic sample. After selecting haplotypes for a given genomic sample, the methylation-genotype-calling system 106 stores reference and target versions of the selected haplotypes as a Positional Burrows Wheeler Transform (PBWT) 812.

As further shown in FIG. 8, in some embodiments, methylation-genotype-calling system 106 samples haplotypes 814 in the PBWT 812 format by performing a linear-time-sampling algorithm based on a haplotype imputation version of HMM developed by Na Li and Matthew Stephens, “Modeling Linkage Disequilibrium and Identifying Recombination Hotspots Using Single-Nucleotide Polymorphism Data,” 165 Genetics 2213-2233 (2003), which is hereby incorporated by reference in its entirety. By performing the linear-time-sampling algorithm as part of sampler iterations, the methylation-genotype-calling system 106 further determines (and updates) the phase of two imputed haplotypes for the genomic coordinate 800 for a target genomic sample.

Based on the imputed and phased haplotypes, as further shown in FIG. 8, the methylation-genotype-calling system 106 determines posterior genotype probabilities 816 that the genomic coordinate 800 of the genomic sample exhibits particular genotypes (e.g., a reference allele or alternate allele). The methylation-genotype-calling system 106 further determines genotype calls for the genomic region for each of the genomic sample. For example, in some implementations, the methylation-genotype-calling system 106 generates the genotype call based on determining a predicted combination of nucleobases corresponding to a highest posterior genotype probability. As indicated above, in some embodiments, the methylation-genotype-calling system 106 uses a modified version of GLIMPSE developed as a genotype imputation model by Rubinacci, S., Ribeiro, D. M., Hofmeister, R. J. et al., “Efficient phasing and imputation of low-coverage sequencing data using large reference panels,” Nat Genet 53, 120-126 (2021) (hereinafter Rubinacci), https://doi.org/10.1038/s41588-020-00756-0, which is hereby incorporated by reference in its entirety. In other embodiments, the methylation-genotype-calling system 106 utilizes a modified HMM model to receive the estimated methylation-level value 820 as input. For example, the methylation-genotype-calling system 106 may utilize a modified version of GLIMPSE as described by Rubinacci.

As indicated above, in some implementations, the methylation-genotype-calling system 106 utilizes prior genotype probabilities as input into an HMM model. As described further below, the methylation-genotype-calling system 106 may (i) determine probabilities of observed bases at a genomic coordinate based on an estimated methylation-level value and base-call-quality metrics and (ii) further determine prior genotype probabilities (as inputs into an HMM model) based on corresponding probabilities of observed bases. FIG. 9 illustrates the methylation-genotype-calling system 106 determining probabilities of observed bases at a genomic coordinate and posterior genotype probabilities for a genomic sample at the genomic coordinate based in part on estimated methylation-level values for observed bases in accordance with one or more embodiments of the present disclosure.

As shown in FIG. 9, the methylation-genotype-calling system 106 determines a probability of an observed base given a haplotype base for each observed nucleobase at the genomic coordinate. The probability of an observed base given a haplotype base can be expressed as P(Rᵢ|Hⱼ), where Rᵢ represents an observed nucleobase and Ha represents a haplotype base.

As shown in FIG. 9, the methylation-genotype-calling system 106 utilizes various equations to determine the probability of an observed base given a haplotype P(Rᵢ|Hⱼ) based on different observed base-haplotype combinations, as follows:

$$P ( {R}_{i} \vert {H}_{j} ) = \{ \begin{matrix}( 1 - \text{?} ) ( 1 - e ) + \text{?} \frac{e}{3} & if ( {R}_{i} , {H}_{j} ) = ( {C}^{+} , C ) \\ ( 1 - \text{?} ) ( 1 - e ) + \text{?} \frac{e}{3} & if ( {R}_{i} , {H}_{j} ) = ( {G}^{-} , G ) \\ \text{?} ( 1 - e ) + ( 1 - \text{?} ) \frac{e}{3} & if ( {R}_{i} , {H}_{j} ) = ( {T}^{+} , C ) \\ \text{?} ( 1 - e ) + ( 1 - \text{?} ) \frac{e}{3} & if ( {R}_{i} , {H}_{j} ) = ( {A}^{-} , G ) \\ 1 - e & else if {R}_{i} = {H}_{j} \\ \frac{e}{3} & else\end{matrix}$$

where [symbol] represents an estimated plus-strand-methylation-level value for an observed base on a plus strand at a genomic coordinate, [symbol] represents an estimated minus-strand-methylation-level value of the observed based on the minus strand at the genomic coordinate, and e represents a probability that the observed base is an error.

As further shown in FIG. 9, in some implementations, for observed base-haplotype base combinations that are not explicitly listed, the methylation-genotype-calling system 106 utilizes the following equation to determine the probability of an observed base:

$$P ( {R}_{i} \vert {H}_{j} ) = \{ \begin{matrix}1 - e & if {R}_{i} = {H}_{j} \\ \frac{e}{3} & else\end{matrix}$$

where e represents a probability that the observed base is an error. In some implementations, the methylation-genotype-calling system 106 determines e based on base-call-quality metrics (e.g., BASEQ score). In one example, e equals 10^{-BASEQ/}10.

As mentioned previously, the methylation-genotype-calling system 106 determines the probability represented by P(Rᵢ|Hⱼ) for each observed nucleobase at the given genomic coordinate. Accordingly, the methylation-genotype-calling system 106 can determine several values for the whole read pileup. The methylation-genotype-calling system 106 further aggregates the P(Rᵢ|Hⱼ) values into a probability of an observed base given a genotype at the genomic coordinate. For example, the probability of an observed base given a genotype can be expressed as P(Rᵢ| [symbol] ), where Rᵢ represents an observed nucleobase and Gx represents the genotype at the genomic coordinate.

As discussed above with relation to FIG. 8, the methylation-genotype-calling system 106 can input the probabilities represented by P(Rᵢ|Hⱼ) or P(Rᵢ| [symbol] ) into a variant caller. In some implementations, and as illustrated in FIG. 9, the variant caller collapses the probabilities into a probability of the observed read pileup, represented as P(D| [symbol] ). The methylation-genotype-calling system 106 can further utilize the variant caller to invert the probability of the observed read pileup to generate posterior genotype probability P( [symbol] |D) where D represents the data or the observed read pileup.

The methylation-genotype-calling system 106 can leverage the posterior genotype probabilities according to P( [symbol] |D) in at least a couple of ways. As indicated above, the methylation-genotype-calling system 106 can determine, from among the posterior genotype probabilities according to P( [symbol] |D) for a given genomic coordinate, a highest posterior genotype probability as the genotype call for the given genomic coordinate. As described further below, such genotype calls outperform the accuracy of existing sequencing and methylation detection systems and approaches the accuracy of non-methylated whole genome sequencing. In addition to genotype calls, the methylation-genotype-calling system 106 can leverage the posterior genotype probabilities according to P( [symbol] |D) to determined refined (and sometimes more accurate) methylation-level values for candidate cytosine bases at target genomic coordinates.

As just indicated, in some implementations, the methylation-genotype-calling system 106 generates a refined methylation-level value for a cytosine base at a genomic coordinate based on posterior genotype probabilities and observed nucleobases at the genomic coordinate. FIG. 10 illustrates the methylation-genotype-calling system 106 generating a refined methylation-level value (\hat{β}) in accordance with one or more embodiments of the present disclosure. In some embodiments, the methylation-genotype-calling system 106 can more accurately predict methylation-level values for a cytosine base at the genomic coordinate based on a more accurate prediction of the genotype.

As shown in FIG. 10, the methylation-genotype-calling system 106 can generate a refined methylation-level value (\hat{β}) utilizing the following equation:

$$\hat{β} = {\sum }_{g ∈ 𝒢} \frac{{\int }_{β}^{} β \times P ( M \vert β , N , g ) P ( β ) d β}{{\int }_{β}^{} P ( M \vert β , N , g ) P ( β ) d β} P ( g \vert D ) = {\sum }_{g ∈ 𝒢} \text{?} \times P ( g \vert D )$$

where M represents a count of methylated nucleobases at the genomic coordinate and N represents a total number of nucleobases at the genomic coordinate. In the equation above, g represents the set of potential genotypes for the genomic coordinate (i.e., CC, CA, CG, and CT). In some implementations, β represents the estimated methylation-level value ( [symbol] ). D represents the data from a read pileup at the genomic coordinate.

As further illustrated in FIG. 10, the methylation-genotype-calling system 106 utilizes different equations to generate the refined methylation-level values based on the observed or predicted genotype. In some embodiments, for example, for predicted genotypes CC, CA, and CG, the methylation-genotype-calling system 106 generates the refined methylation-level value utilizing the following equation:

$$\text{?} = \text{?} = \text{?} = \frac{M + a}{N + a + b}$$

where M represents a count of methylated nucleobases at the genomic coordinate and N represents a total number of observed nucleobases at the genomic coordinate. In the equation above, a and b represent tuneable parameters utilized in the following beta prior function for the estimated methylation-level value:

$$P ( β ) = \frac{{{β}^{a - 1} ( 1 - β )}^{b - 1}}{Beta ( a , b )}$$

For predicted a predicted genotype of CT, by contrast, the methylation-genotype-calling system 106 can utilize the following equation to generate the refined methylation-level value:

$$\text{?} = \frac{a}{N - M + a + b} \frac{{}_{2}^{} {F}_{1} ( - M , a + 1 ; N - M + a + b + 1 ; - 1 )}{{}_{2}^{} {F}_{1} ( - M , a ; N - M + a + b ; - 1 )}$$

Where M represents a count of methylated nucleobases at the genomic coordinate and N represents a total number of observed nucleobases at the genomic coordinate. In the equation above, a and b represent tuneable parameters utilized in the above-mentioned beta prior function. Additionally, and as shown in FIG. 10, [symbol] (a, b; c; x) represents a hypergeometric function.

As mentioned, in some examples, the refined methylation-level value (\hat{β}) is more accurate than the estimated methylation-level value ( [symbol] ). In particular, the methylation-genotype-calling system 106 improves the accuracy of the refined methylation-level value by computing different methylation-level value estimates and weighting the methylation-level value estimates based on the posterior genotype probability. For example, only one of the subset of candidate genotypes (CC, CA, CG, or CT) for a given genomic coordinate is accurate. Based on posterior genotype probabilities that indicate a strong likelihood of a genotype CC, for example, the methylation-genotype-calling system 106 can collapse the whole expression and heavily weight the final refined methylation-level value using [symbol] . For example, in some implementations, based on determining that a posterior genotype probability for a given genotype is above a threshold value, the methylation-genotype-calling system 106 may determine the refined methylation-level value based on the equation specific to the given genotype. The methylation-genotype-calling system 106 may also allow for a mixed weighting of expressions if the genotype is uncertain based on the posterior genotype probabilities.

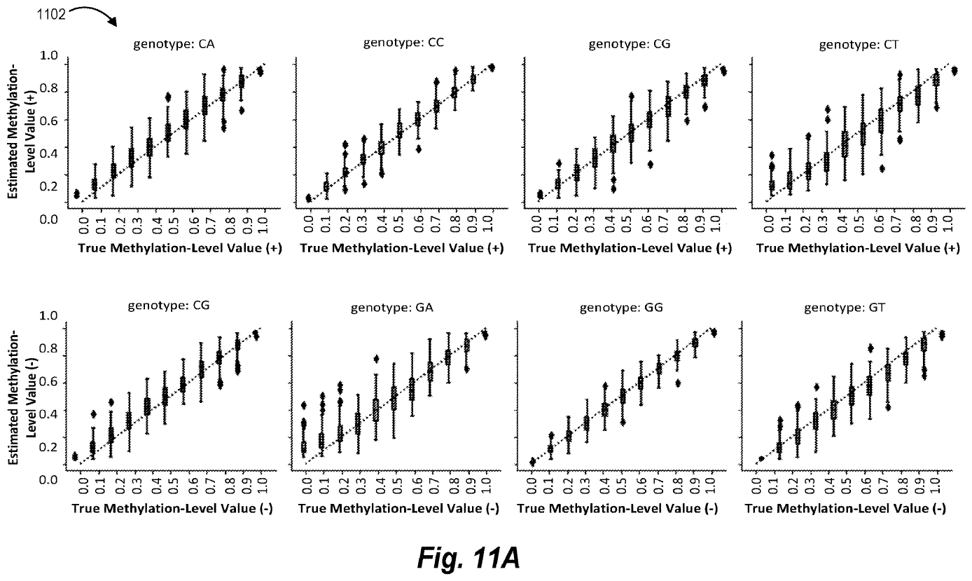

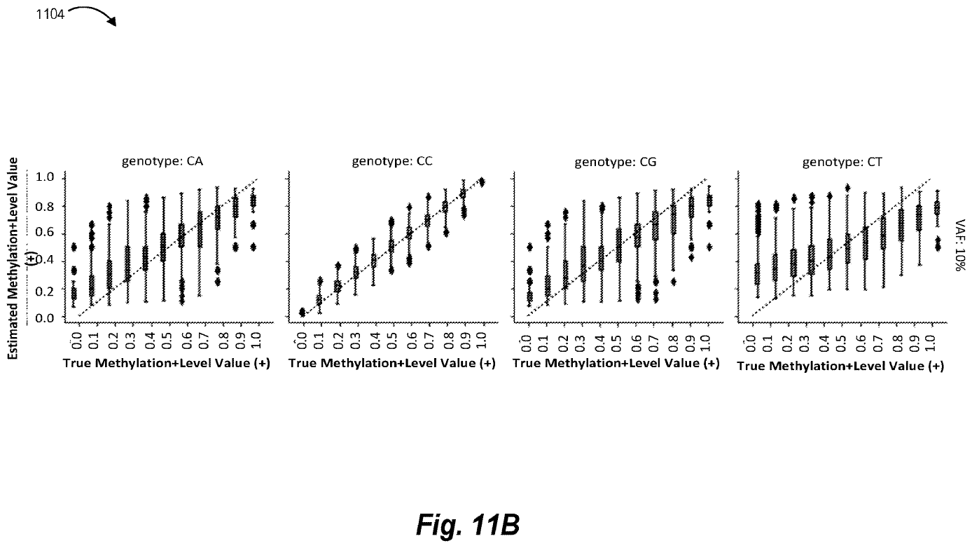

As mentioned, the methylation-genotype-calling system 106 improves the accuracy of both methylation-level-value estimations and genotype calls relative to existing sequencing and methylation detection systems. FIGS. 11A-13C illustrate various graphs and charts portraying such improvements in accuracy made by the methylation-genotype-calling system 106.

FIGS. 11A-11B illustrate graphs demonstrating improvements made by the methylation-genotype-calling system 106 in accurately predicting methylation-level values. More particularly, FIG. 11A illustrates the methylation-genotype-calling system 106 accurately estimating methylation-level values for cytosine bases on plus and minus strands for a germline allele. FIG. 11B illustrates the methylation-genotype-calling system 106 accurately estimating methylation-level values for cytosine bases on plus strands with a 10% C allele frequency. While the graphs in FIGS. 11A-11B depict results from simulated data, where observed nucleotide reads are simulated for given genotypes, the methylation-genotype-calling system 106 would necessarily generate the more accurate, estimated methylation-level values in the graphs of FIGS. 11A-11B using the equations described above to determine refined methylation-level values. Accordingly, the type of estimated methylation-level values depicted in graphs of FIGS. 11A-11B are refined methylation-level values.

In particular, FIG. 11A illustrates graphs 1102 that portray estimated methylation-level values generated by the methylation-genotype-calling system 106 compared to true methylation-level values. More specifically, the graphs 1102 reflect the accuracy of estimated methylation-level values generated by the methylation-genotype-calling system 106 for cytosine bases at a genomic coordinate on both plus and minus strands for a germline allele. As shown in FIG. 11A, for depicted genotypes of CA, CC, CG, and CT on the plus strand, and CG, GA, GG, and GT on the minus strand, the estimated methylation-level values closely track the true methylation-level values illustrated by the dotted lines. For example, as discussed above with relation to FIG. 6, in contrast with existing systems that often inaccurately estimate methylation-level values for CA, CT, and CG genotypes. But the methylation-genotype-calling system 106 accurately estimates methylation-level values for these same genotypes as shown in FIG. 11A.

FIG. 11B illustrates graphs 1104 that portray estimated methylation-level values generated by the methylation-genotype-calling system 106 compared to true methylation-level values from a somatic allele. More specifically, the graphs 1104 portray the accuracy of methylation-level values generated by the methylation-genotype-calling system 106 for a genomic sample with somatic variants having 10% variant allele frequency (VAF) of the cytosine allele. As shown, the estimated methylation-level values generated by the methylation-genotype-calling system 106 closely track the true methylation-level values indicated by the dotted lines. Though the graphs illustrated in FIG. 11B corresponding to CA, CT, and CG genotypes portray a wider range of variation in results, the estimated methylation-level values generated by the methylation-genotype-calling system 106 are more accurate than estimated methylation-level values predicted by existing sequencing and methylation detection systems described above. In fact, many existing sequencing and methylation detection systems that rely on SBS technology are wholly incapable of predicting methylation levels from a somatic allele.

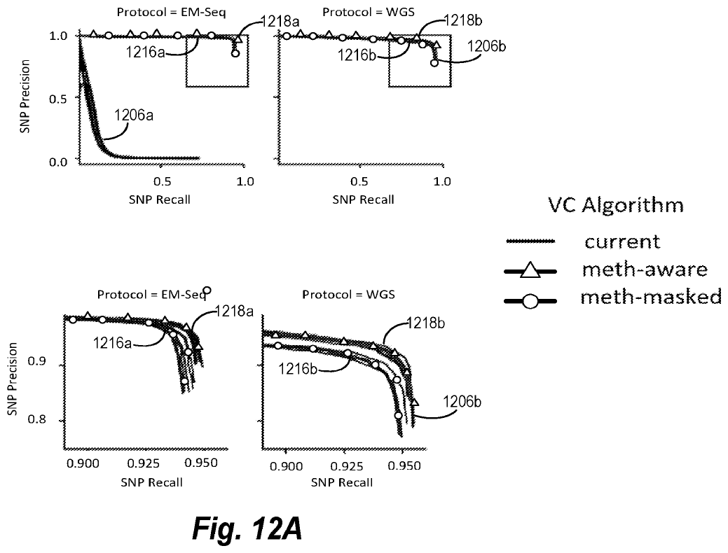

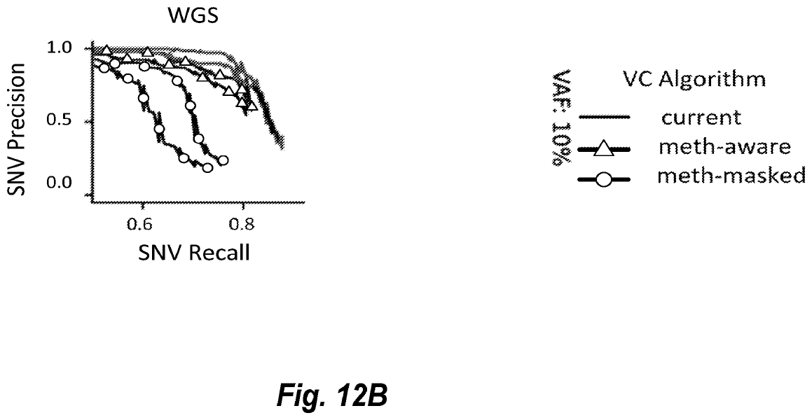

In addition to generating more accurate methylation-level values, the methylation-genotype-calling system 106 also more accurately calls single nucleotide polymorphisms (SNPs). FIGS. 12A-12B illustrate a series of plots demonstrating that the methylation-genotype-calling system 106 more accurately calls SNPs relative to existing sequencing and methylation detection systems in accordance with one or more embodiments of the present disclosure.

FIGS. 12A-12B illustrate various plots that map SNP precision and SNP recall for various variant caller (VC) algorithms. For instance, the plots map results from a current VC algorithm, a methylation-aware VC algorithm (meth-aware), and a methylation-masked VC algorithm (meth-masked). The current VC algorithm data represents a basic variant caller utilized by existing methylation and detection systems, such as the model employed in FIG. 5. Methylation-aware VC algorithm data represents results generated by the methylation-genotype-calling system 106 in accordance with one or more embodiments of the present disclosure. Methylation-masked VC algorithm data represents results generated by a more complicated and more accurate variant caller than the “current” VC. For example, the methylation-masked VC algorithm may improve SNP precision and SNP recall relative to a current VC algorithm by accounting for methylation conversions. More specifically, the methylation-masked VC algorithm reduces values of prior genotype probabilities for variant calls corresponding to nucleobases converted by a methylation sequencing assay. For example, the methylation-masked VC algorithm assigns lower quality scores (e.g., Q-score of 0) to thymine bases on a plus strand or adenine bases on a minus strand.

As shown in FIG. 12A, the methylation-genotype-calling system 106 improves both SNP precision and recall relative to existing sequencing and methylation detection systems. More particularly, FIG. 12A illustrates two graphs and enriched regions of those graphs. The graphs show results for an EM-Seq protocol and a whole genome sequencing (WGS) protocol. Results of the methylation-genotype-calling system 106 are shown by lines 1218a-1218b on the plots illustrated in FIG. 12A. Lines 1206a-1206b reflect the performance of the current VC algorithm; lines 1216a-1216b reflect the performance of the methylation-genotype-calling system 106, which can likewise be referred to as the methylation-aware VC algorithm; and lines 1218a-1218b reflect the performance of the methylation-masked VC algorithm.

For the EM-Seq protocol, and as shown by the line 1206a, the current VC algorithm corresponds with a drop in both precision and recall. Both the methylation-genotype-calling system 106 and methylation-masked VC algorithm show improved performance in an EM-Seq protocol relative to existing sequencing and methylation detection systems. For example, as shown by the lines 1218a-1218b, the methylation-genotype-calling system 106 performs similarly and to a WGS algorithm in both SNP precision and recall.

The methylation-genotype-calling system 106 may also accurately predict single nucleotide variants (SNV) in somatic variant calling. FIG. 12B illustrates the methylation-genotype-calling system 106 accurately calling SNVs in somatic variant calling. As illustrated by FIG. 12B, the methylation-genotype-calling system 106 is one of the first known systems to accurately call somatic variants from short-read methylation sequencing data. Lines 1222a and 1222b represent somatic-variant-calling performance of the methylation-genotype-calling system 106 (meth-aware) for different samples NA12877 and NA12878, where 10% of NA12877 has been added in to NA12878 at TruSight One (TSO) enriched genomic regions at 300× coverage. By contrast, lines 1224a and 1224b represent somatic-variant-calling performance of the current VC algorithm for different samples NA12877 and NA12878, where 10% of NA12877 has likewise been added in to NA12878 at TSO enriched genomic regions at 300× coverage. Further, lines 1220a and 1220b represent somatic-variant-calling performance of a meth-masked VC algorithm for different samples NA12877 and NA12878, where 10% of NA12877 has likewise been added in to NA12878 at TSO enriched genomic regions at 300× coverage. As shown in both FIGS. 12A and 12B, the methylation-genotype-calling system 106 performs at both higher SNV precision and recall relative to meth-masked methods. The methylation-genotype-calling system 106 also matches current VC methods for WGS protocols.

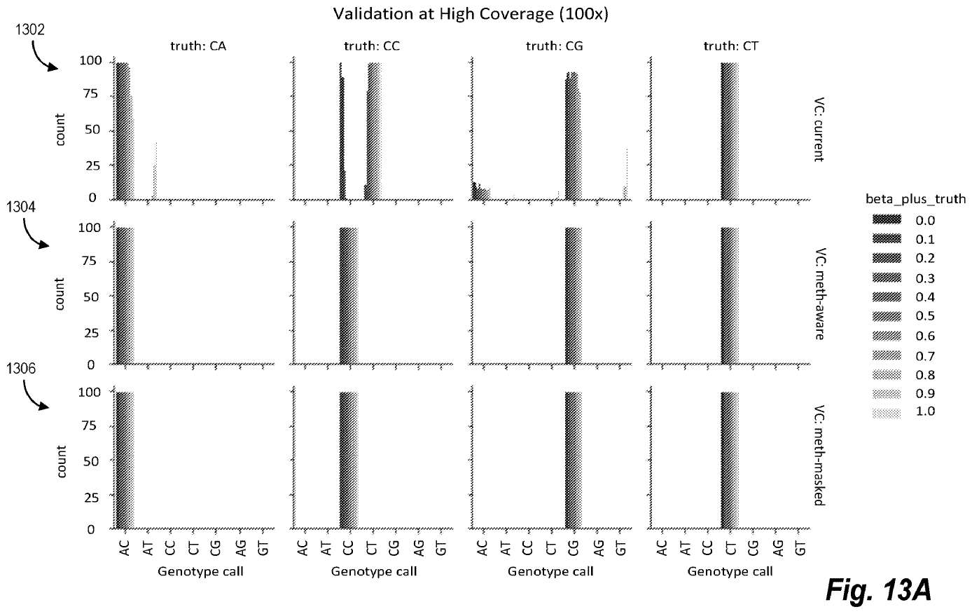

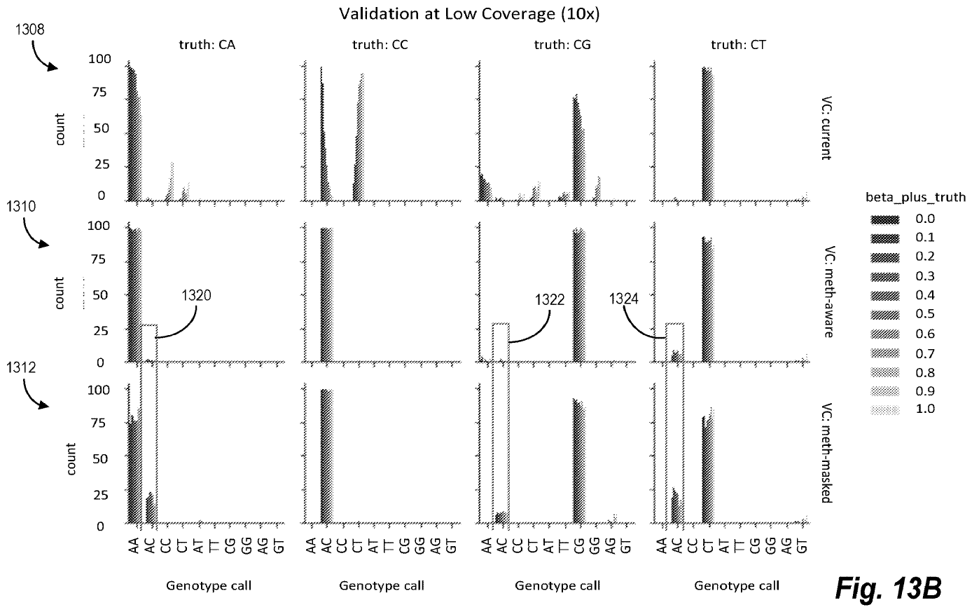

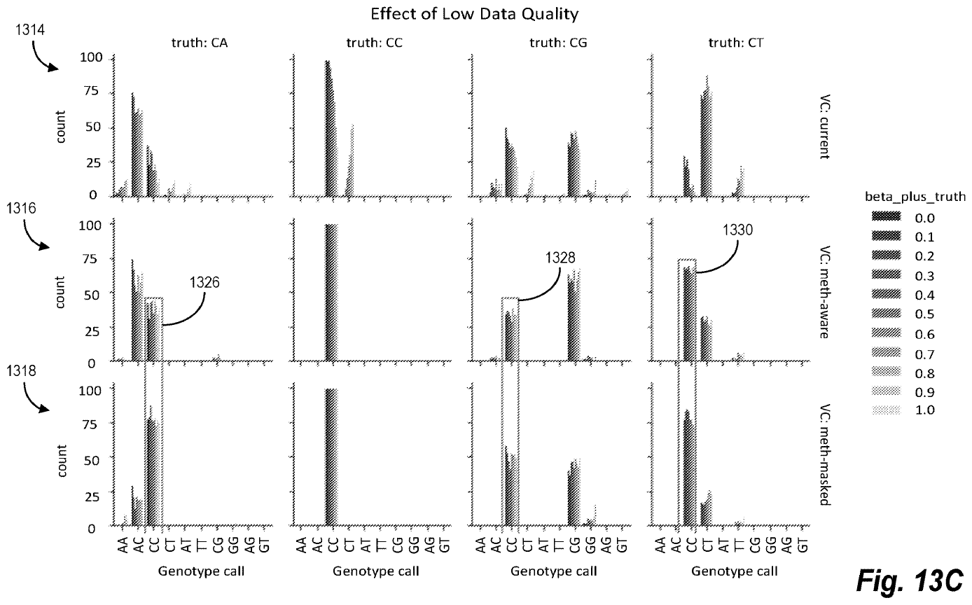

As discussed previously, the methylation-genotype-calling system 106 can more accurately call genotypes than existing sequencing and methylation detection systems that perform C>T conversion-based sequencing. FIGS. 13A-13C illustrate a series of charts demonstrating that the methylation-genotype-calling system 106 accurately calls genotypes at genomic coordinates in accordance with one or more embodiments of the present disclosure. FIGS. 13A-13C illustrate the accuracy of genotype calls generated by the methylation-genotype-calling system 106 (methylation-aware) relative to methylation-masked and current VC protocols under different conditions. For example, FIG. 13A illustrates effects of high coverage for genomic regions with 100× read coverage, FIG. 13B illustrates effects at low coverage for genomic regions with 10× read coverage, and FIG. 13C illustrates effects of low data quality.

FIG. 13A shows the effect of high coverage (100×) on validation. As just suggested, coverage describes the average number of reads that align to or cover known reference bases. Charts 1302 show validation results for current VC protocols, charts 1304 show validation results for the methylation-genotype-calling system 106, and charts 1306 show validation results for methylation-masked VC protocols. As shown in FIG. 13A, the methylation-genotype-calling system 106 and methylation-masked VC protocols both call genotypes more accurately than current VC protocols. Current VC protocols refer to existing sequencing and methylation detection systems. For example, current VC protocols often miscall genotypes where the true genotype is CA, CC, or CG.

FIG. 13B shows the effect of low coverage (10×) on validation. Charts 1308 show validation results for current VC protocols, charts 1310 show validation results for the methylation-genotype-calling system 106, and charts 1312 show validation results for methylation-masked VC protocols. As shown in FIG. 13B, current VC protocols are negatively impacted and miscall more genotypes at low coverage. The methylation-genotype-calling system 106 more accurately calls CA and CG genotypes than methylation-masked VC protocols as shown by boxes 1320 and 1322, respectively. Furthermore, as shown by boxes 1324, while the methylation-genotype-calling system 106 may miscall CT genotypes, the number of miscalled bases is less than those miscalled by methylation-masked VC protocols. Thus, the methylation-genotype-calling system 106 outperforms existing systems in validation-even at low coverage.

FIG. 13C shows the effect of low-quality data on validation. Charts 1314 show validation results for current VC protocols, charts 1316 show validation results for the methylation-genotype-calling system 106, and charts 1318 show validation results for methylation-masked VC protocols. While FIGS. 13A-13B show results corresponding with data having a BASEQ score of 40, the data for FIG. 13C has a BASEQ score of 10. With low quality data, and as shown by highlight 1326 and highlight 1328, the methylation-genotype-calling system 106 calls CA and CG genotypes more accurately than methylation-masked VC protocols. As shown by highlight 1330, the methylation-genotype-calling system 106 often miscalls true CT genotypes as CC. However, overall, the methylation-genotype-calling system 106 more accurately calls most genotypes than current and methylation-masked VC systems.

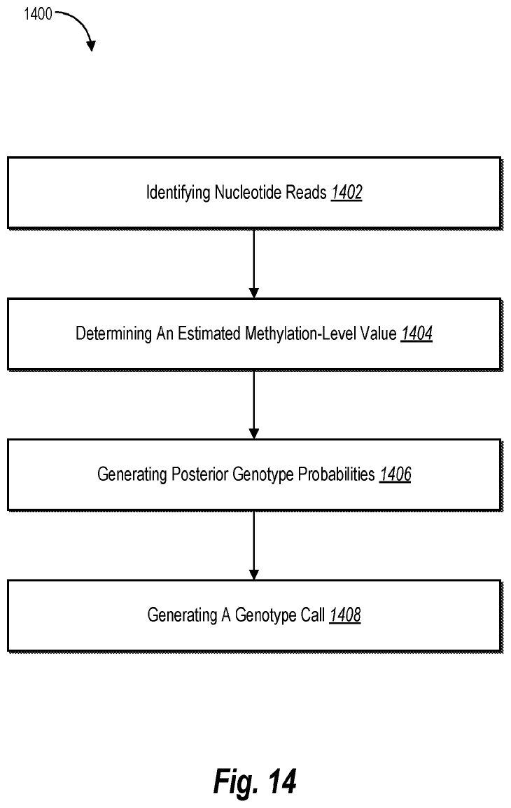

FIGS. 1-13C, the corresponding text, and the examples provide a number of different methods, systems, devices, and non-transitory computer-readable media of the methylation-genotype-calling system 106. In addition to the foregoing, one or more implementations can also be described in terms of flowcharts comprising acts for accomplishing a particular result, as shown in FIG. 14. FIG. 14 illustrates a flowchart of a series of acts 1400 of generating a genotype call and an estimated methylation-level value in accordance with one or more embodiments of the present disclosure. While FIG. 14 illustrates acts according to one embodiment, alternative embodiments may omit, add to, reorder, and/or modify any of the acts shown in FIG. 14. The acts of FIG. 14 can be performed as part of a method. Alternatively, a non-transitory computer readable storage medium can comprise instructions that, when executed by one or more processors, cause a computing device or a system to perform the acts depicted in FIG. 14. In still further embodiments, a system comprising at least one processor and a non-transitory computer readable medium comprising instructions that, when executed by one or more processors, cause the system to perform the acts of FIG. 14.

As shown in FIG. 14, the series of acts 1400 includes an act 1402 of identifying nucleotide reads, an act 1404 of determining an estimated methylation-level value, an act 1406 of generating posterior genotype probabilities, and an act 1408 of generating a genotype call. For example, the series of acts 1400 can include acts to perform any of the operations described in the following clauses:

CLAUSE 1. A method comprising:
identifying, for a target genomic sample, nucleotide reads comprising one or more nucleobases converted by a methylation sequencing assay;
determining an estimated methylation-level value for a cytosine base at a genomic coordinate based on prior genotype probabilities for the target genomic sample at the genomic coordinate and observed nucleobases at the genomic coordinate within the nucleotide reads;
generating, utilizing a variant call model, posterior genotype probabilities for the target genomic sample at the genomic coordinate based on the estimated methylation-level value and base-call-quality metrics for the observed nucleobases; and generating, based on the posterior genotype probabilities, a genotype call that the target genomic sample comprises a predicted combination of nucleobases at the genomic coordinate.

CLAUSE 2. The method of clause 1, further comprising generating a refined methylation-level value for the cytosine base at the genomic coordinate based on the posterior genotype probabilities and the observed nucleobases.

CLAUSE 3. The method of clause 2, further comprising generating the refined methylation-level value by:
determining genotype-specific methylation-level values corresponding with each possible genotype at the genomic coordinate based on the observed nucleobases; and
weighting the genotype-specific methylation-level values based on the posterior genotype probabilities.

CLAUSE 4. The method of clause 1, wherein generating the genotype call for the target genomic sample is further based on sequencing metrics corresponding to the nucleotide reads.

CLAUSE 5. The method of clause 1, wherein the posterior genotype probabilities comprise a subset of posterior genotype probabilities for cytosine base in a plus strand or a minus strand.

CLAUSE 6. The method of clause 1, further comprising determining the estimated methylation-level value by:
determining a probability of each nucleobase at the genomic coordinate on a plus strand and a minus strand;
determining probabilities of the observed nucleobases at the genomic coordinate based on numbers of each observed nucleobase, the probability of each nucleobase at the genomic coordinate, and the prior genotype probabilities; and
generating an estimated plus-strand-methylation-level value for a nucleobase at the genomic coordinate on the plus strand and an estimated minus-strand-methylation-level value for a nucleobase at the genomic coordinate on the minus strand by performing a Bayesian inversion on the probabilities of the observed nucleobases.

CLAUSE 7. The method of clause 6, further comprising determining the probability of each nucleobase by:
determining that a probability of a given thymine base on the plus strand approximates the estimated methylation-level value; and
determining that a probability of a given cytosine base on the plus strand approximates one minus the estimated methylation-level value.

CLAUSE 8. The method of clause 6, further comprising determining the probability of each nucleobase by:
determining that a probability of a given adenine base on the minus strand approximates the estimated methylation-level value; and
determining that a probability of a given guanine base on the minus strand approximates one minus the estimated methylation-level value.

CLAUSE 9. The method of clause 1, wherein the variant call model comprises a Hidden Markov model (HMM) modified to receive an input based the estimated methylation-level value and a base-call-quality metric for a corresponding observed nucleobase.

CLAUSE 10. The method of clause 1, further comprising generating the genotype call based on determining a predicted combination of nucleobases corresponding to a highest posterior genotype probability.

CLAUSE 11. The method of clause 1, wherein identifying the nucleotide reads comprising the one or more nucleobases converted by the methylation sequencing assay comprises identifying the nucleotide reads comprising thymine bases or uracil bases converted from cytosine bases by the methylation sequencing assay.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the methylation-genotype-calling system 106 can include software, hardware, or both. For example, the components of the methylation-genotype-calling system 106 can include one or more instructions stored on a computer-readable storage medium and executable by processors of one or more computing devices (e.g., the user client device 110). When executed by the one or more processors, the computer-executable instructions of the methylation-genotype-calling system 106 can cause the computing devices to perform the bubble detection methods described herein. Alternatively, the components of the methylation-genotype-calling system 106 can comprise hardware, such as special purpose processing devices to perform a certain function or group of functions. Additionally, or alternatively, the components of the methylation-genotype-calling system 106 can include a combination of computer-executable instructions and hardware.

Furthermore, the components of the methylation-genotype-calling system 106 performing the functions described herein with respect to the methylation-genotype-calling system 106 may, for example, be implemented as part of a stand-alone application, as a module of an application, as a plug-in for applications, as a library function or functions that may be called by other applications, and/or as a cloud-computing model. Thus, components of the methylation-genotype-calling system 106 may be implemented as part of a stand-alone application on a personal computing device or a mobile device. Additionally, or alternatively, the components of the methylation-genotype-calling system 106 may be implemented in any application that provides sequencing services including, but not limited to Illumina BaseSpace, Illumina DRAGEN, Illumina NextSeq, Illumina TruSeq, or Illumina TruSight software. “Illumina,” “BaseSpace,” “DRAGEN,” “NextSeq,” “TruSeq,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 15 illustrates a block diagram of a computing device 1500 that may be configured to perform one or more of the processes described above. One will appreciate that one or more computing devices such as the computing device 1500 may implement the methylation-genotype-calling system 106. As shown by FIG. 15, the computing device 1500 can comprise a processor 1502, a memory 1504, a storage device 1506, an I/O interface 1508, and a communication interface 1510, which may be communicatively coupled by way of a communication infrastructure 1512. In certain embodiments, the computing device 1500 can include fewer or more components than those shown in FIG. 15. The following paragraphs describe components of the computing device 1500 shown in FIG. 15 in additional detail.

In one or more embodiments, the processor 1502 includes hardware for executing instructions, such as those making up a computer program. As an example, and not by way of limitation, to execute instructions for dynamically modifying workflows, the processor 1502 may retrieve (or fetch) the instructions from an internal register, an internal cache, the memory 1504, or the storage device 1506 and decode and execute them. The memory 1504 may be a volatile or non-volatile memory used for storing data, metadata, and programs for execution by the processor(s). The storage device 1506 includes storage, such as a hard disk, flash disk drive, or other digital storage device, for storing data or instructions for performing the methods described herein.

The I/O interface 1508 allows a user to provide input to, receive output from, and otherwise transfer data to and receive data from computing device 1500. The I/O interface 1508 may include a mouse, a keypad or a keyboard, a touch screen, a camera, an optical scanner, network interface, modem, other known I/O devices or a combination of such I/O interfaces. The I/O interface 1508 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1508 is configured to provide graphical data to a display for presentation to a user. The graphical data may be representative of one or more graphical user interfaces and/or any other graphical content as may serve a particular implementation.

The communication interface 1510 can include hardware, software, or both. In any event, the communication interface 1510 can provide one or more interfaces for communication (such as, for example, packet-based communication) between the computing device 1500 and one or more other computing devices or networks. As an example, and not by way of limitation, the communication interface 1510 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1510 may facilitate communications with various types of wired or wireless networks. The communication interface 1510 may also facilitate communications using various communication protocols. The communication infrastructure 1512 may also include hardware, software, or both that couples components of the computing device 1500 to each other. For example, the communication interface 1510 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

1. A computer-implemented method comprising:
identifying, for a target genomic sample, nucleotide reads comprising one or more nucleobases converted by a methylation sequencing assay;
determining an estimated methylation-level value for a cytosine base at a genomic coordinate based on prior genotype probabilities for the target genomic sample at the genomic coordinate and observed nucleobases at the genomic coordinate within the nucleotide reads;
generating, utilizing a variant call model, posterior genotype probabilities for the target genomic sample at the genomic coordinate based on the estimated methylation-level value and base-call-quality metrics for the observed nucleobases; and
generating, based on the posterior genotype probabilities, a genotype call that the target genomic sample comprises a predicted combination of nucleobases at the genomic coordinate.

2. The computer-implemented method of claim 1, further comprising generating a refined methylation-level value for the cytosine base at the genomic coordinate based on the posterior genotype probabilities and the observed nucleobases.

3. The computer-implemented method of claim 2, further comprising generating the refined methylation-level value by:
determining genotype-specific methylation-level values corresponding with each possible genotype at the genomic coordinate based on the observed nucleobases; and
weighting the genotype-specific methylation-level values based on the posterior genotype probabilities.

4. The computer-implemented method of claim 1, wherein generating the genotype call for the target genomic sample is further based on sequencing metrics corresponding to the nucleotide reads.

5. The computer-implemented method of claim 1, wherein the posterior genotype probabilities comprise a subset of posterior genotype probabilities for cytosine base in a plus strand or a minus strand.

6-11. (canceled)

12. A system comprising:
at least one processor; and
a non-transitory computer-readable medium comprising instructions that, when executed by the at least one processor, cause the system to:
identify, for a target genomic sample, nucleotide reads comprising one or more nucleobases converted by a methylation sequencing assay;
determine an estimated methylation-level value for a cytosine base at a genomic coordinate based on prior genotype probabilities for the target genomic sample at the genomic coordinate and observed nucleobases at the genomic coordinate within the nucleotide reads;
generate, utilizing a variant call model, posterior genotype probabilities for the target genomic sample at the genomic coordinate based on the estimated methylation-level value and base-call-quality metrics for the observed nucleobases; and
generate, based on the posterior genotype probabilities, a genotype call that the target genomic sample comprises a predicted combination of nucleobases at the genomic coordinate.

13. The system of claim 12, further comprising instructions that, when executed by the at least one processor, cause the system to generate a refined methylation-level value for the cytosine base at the genomic coordinate based on the posterior genotype probabilities and the observed nucleobases.

14. The system of claim 13, further comprising instructions that, when executed by the at least one processor, cause the system to generate the refined methylation-level value by:
determining genotype-specific methylation-level values corresponding with each possible genotype at the genomic coordinate based on the observed nucleobases; and
weighting the genotype-specific methylation-level values based on the posterior genotype probabilities.

15. The system of claim 12, further comprising instructions that, when executed by the at least one processor, cause the system to generate the genotype call for the target genomic sample further based on sequencing metrics corresponding to the nucleotide reads.

16. The system of claim 12, wherein the posterior genotype probabilities comprise a subset of posterior genotype probabilities for cytosine base in a plus strand or a minus strand.

17. The system of claim 12, further comprising instructions that, when executed by the at least one processor, cause the system to determine the estimated methylation-level value by:
determining a probability of each nucleobase at the genomic coordinate on a plus strand and a minus strand;
determining probabilities of the observed nucleobases at the genomic coordinate based on numbers of each observed nucleobase, the probability of each nucleobase at the genomic coordinate, and the prior genotype probabilities; and
generating an estimated plus-strand methylation-level value for a nucleobase at the genomic coordinate on the plus strand and an estimated minus-strand methylation-level value for a nucleobase at the genomic coordinate on the minus strand by performing a Bayesian inversion on the probabilities of the observed nucleobases.

18. The system of claim 17, further comprising instructions that, when executed by the at least one processor, cause the system to determine the probability of each nucleobase by:
determining that a probability of a given thymine base on the plus strand approximates the estimated methylation-level value; and
determining that a probability of a given cytosine base on the plus strand approximates one minus the estimated methylation-level value.

19. The system of claim 17, further comprising instructions that, when executed by the at least one processor, cause the system to determine the probability of each nucleobase by:
determining that a probability of a given adenine base on the minus strand approximates the estimated methylation-level value; and
determining that a probability of a given guanine base on the minus strand approximates one minus the estimated methylation-level value.

20. The system of claim 12, wherein the variant call model comprises a Hidden Markov model (HMM) modified to receive an input based on the estimated methylation-level value and a base-call-quality metric for a corresponding observed nucleobase.

21. The system of claim 12, further comprising instructions that, when executed by the at least one processor, cause the system to generate the genotype call based on determining a predicted combination of nucleobases corresponding to a highest posterior genotype probability.

22. The system of claim 12, further comprising instructions that, when executed by the at least one processor, cause the system to identify the nucleotide reads comprising the one or more nucleobases converted by the methylation sequencing assay by identifying the nucleotide reads comprising thymine bases or uracil bases converted from cytosine bases by the methylation sequencing assay.

23. A non-transitory computer-readable medium storing instructions that, when executed by at least one processor, cause a computing device to:
identify, for a target genomic sample, nucleotide reads comprising one or more nucleobases converted by a methylation sequencing assay;
determine an estimated methylation-level value for a cytosine base at a genomic coordinate based on prior genotype probabilities for the target genomic sample at the genomic coordinate and observed nucleobases at the genomic coordinate within the nucleotide reads;
generate, utilizing a variant call model, posterior genotype probabilities for the target genomic sample at the genomic coordinate based on the estimated methylation-level value and base-call-quality metrics for the observed nucleobases; and
generate, based on the posterior genotype probabilities, a genotype call that the target genomic sample comprises a predicted combination of nucleobases at the genomic coordinate.

24. The non-transitory computer-readable medium of claim 23, further comprising instructions that, when executed by the at least one processor, cause the computing device to determine the estimated methylation-level value by:
determining a probability of each nucleobase at the genomic coordinate on a plus strand and a minus strand;
determining probabilities of the observed nucleobases at the genomic coordinate based on numbers of each observed nucleobase, the probability of each nucleobase at the genomic coordinate, and the prior genotype probabilities; and
generating an estimated plus-strand methylation-level value for a nucleobase at the genomic coordinate on the plus strand and an estimated minus-strand methylation-level value for a nucleobase at the genomic coordinate on the minus strand by performing a Bayesian inversion on the probabilities of the observed nucleobases.

25. The non-transitory computer-readable medium of claim 23, wherein the variant call model comprises a Hidden Markov model (HMM) modified to receive an input based on the estimated methylation-level value and a base-call-quality metric for a corresponding observed nucleobase.

26. The non-transitory computer-readable medium of claim 23, further comprising instructions that, when executed by the at least one processor, cause the computing device to generate the genotype call based on determining a predicted combination of nucleobases corresponding to a highest posterior genotype probability.

27. (canceled)
